(19) United States
(12) Patent Application Publication
(10) Pub. No.: US 2023/0341397 A1
(43) Pub. Date: Oct. 26, 2023

(54) PROTEASE BIOSENSORS AND METHODS OF VIRUS DETECTION

(71) Applicant: Montana Molecular LLC, Bozeman, MT (US)

(72) Inventor: Thomas E. HUGHES, Bozeman, MT (US)

(21) Appl. No.: 18/182,030

(22) Filed: Mar. 10, 2023

Related U.S. Application Data
(63) Continuation of application No. PCT/US21/71431, filed on Sep. 10, 2021.
(60) Provisional application No. 63/077,096, filed on Sep. 11, 2020.

(51) Int. Cl. G01N 33/569; C12N 9/50

(52) U.S. Cl. CPC G01N 33/56983; C12N 9/503; C12Y 304/22069; G01N 2333/165; G01N 2333/9506; G01N 2500/04; G01N 2500/10

(57) ABSTRACT

The present disclosure provides a biosensor for the detection of protease activity. The detection of protease activity can be used for the detection of viral infection, in particular coronavirus infection. The biosensor described herein can be used to detect SARS-CoV-2. The present disclosure also provides vectors expressing the biosensor and methods for using the same.

### RELATED APPLICATIONS

This application is a continuation of International Patent Application No. PCT/US2021/071431, filed on Sep. 10, 2021, which claims priority to U.S. Provisional Patent Application No. 63/077,096, filed on Sep. 11, 2020, each of which is incorporated herein by reference in its entirety.

### INCORPORATION BY REFERENCE OF SEQUENCE LISTING

This application contains a Sequence Listing in computer readable form (filename: 404354-006US_197984_Substitute_SL.xml; 57,953 bytes; created Jul. 12, 2023), which is incorporated herein by reference in its entirety and forms part of the disclosure.

### FIELD

The present invention relates to the fields of medicine, cell biology, molecular biology and genetics. In particular, the present invention relates to protease biosensors and their use in detecting viruses.

### BACKGROUND

A common design for protease biosensors involves placing a protease cleavage site in between two reporter proteins that are undergoing Forster Resonance Energy Transfer (FRET). Current caspase sensors that detect apoptosis are excellent examples of this design. A blue and green fluorescent protein pair (Xu, X., et al. 1998. “Detection of Programmed Cell Death Using Fluorescence Energy Transfer.” Nucleic Acids Research 26 (8): 2034-35), or a green and red pair (Shcherbo, et al. 2009. “Practical and Reliable FRET/FLIM Pair of Fluorescent Proteins.” BMC Biotechnology 9 (1): 24; Kawai, et al. 2005. “Simultaneous Real-Time Detection of Initiator- and Effector-Caspase Activation by Double Fluorescence Resonance Energy Transfer Analysis.” Journal of Pharmacological Sciences 97 (3): 361-68) can be connected with a protein linker that contains a caspase cleavage site. The presence of the activated caspase protease cleaves this linker, and the average distance between the two fluorescent proteins rapidly increases. Because the efficiency of FRET is exquisitely sensitive to differences in the distance and orientation of the two fluorophores (Stryer, L., and R. P. Haugland. 1967. “Energy Transfer: A Spectroscopic Ruler.” Proceedings of the National Academy of Sciences of the United States of America 58 (2): 719-26), the cleavage of the linker generates a change in FRET efficiency. Similarly, Bioluminescent Energy Transfer (BRET) can be used in this design, where protease cleavage would produce a change in energy transfer (Xu, Y., D. W. Piston, and C. H. Johnson. 1999. “A Bioluminescence Resonance Energy Transfer (BRET) System: Application to Interacting Circadian Clock Proteins.” Proceedings of the National Academy of Sciences of the United States of America 96 (1): 151-56; Hamer, Anniek den, et al. 2017. “Bright Bioluminescent BRET Sensor Proteins for Measuring Intracellular Caspase Activity.” ACS Sensors 2 (6): 729-34).

Another common design for fluorescent proteins are those that depend on protein complementation and dimerization. Reporter proteins can be split into two complementing fragments, and protease cleavage sensors have been constructed in which complementation between fragments is constrained until cleavage occurs (Zhang, Qiang, et al. 2019. “Designing a Green Fluorogenic Protease Reporter by Flipping a Beta Strand of GFP for Imaging Apoptosis in Animals.” Journal of the American Chemical Society, March).

Fluorescent proteins can also be designed to fluoresce as a function of protein degradation. The amino acid at the N-terminus of a mature protein often defines the half-life of the protein (Bachmair, A., D. Finley, and A. Varshaysky. 1986. “In Vivo Half-Life of a Protein Is a Function of Its Amino-Terminal Residue.” Science 234 (4773): 179-86). This is known as the N-end rule. Ubiquitination often controls the degradation rate of a protein, and ubiquitination enzymes can fuse ubiquitin to the N-terminus of a protein, as well as to lysine residues in the protein. When de-ubiquitination enzymes cleave ubiquitin added to the N-terminus of a protein, this exposes a new N-terminus. Because of the N-end rule, this new N-terminus defines the half-life of the remaining protein (Varshaysky, Alexander. 2019. “N-Degron and C-Degron Pathways of Protein Degradation.” Proceedings of the National Academy of Sciences of the United States of America 116 (2): 358-66). Ubiquitin has been added to the N-terminus of proteins, followed by particular amino acids, to destabilize the protein by exposing new N-termini that, according to the N-end rule, shorten the half-life of the protein. Positioning ubiquitin in such a manner is known as “destabilizing” a protein, and the ubiquitin domain is referred to as a “degron.” Ubiquitin-based degrons have been added to fluorescent proteins to shorten their half-life, or destabilize them (Houser, John R., et al. 2012. “An Improved Short-Lived Fluorescent Protein Transcriptional Reporter for Saccharomyces cerevisiae.” Yeast 29 (12): 519-30).

SARS-CoV-2 is an emerging global health crisis with over 25 million reported cases to date. As the SARS-CoV-2 pandemic continues to expand, intense efforts from both academia and industry are focused on the development of vaccines or treatments to ameliorate symptoms and eventually, stop the virus transmission. Thus, there is a need for biosensor specific to SARS-CoV-2 to detect SARS-CoV-2 replication and compounds that can inhibit the same.

### SUMMARY

The disclosure provides compositions and methods for protease biosensors and their use in detecting protease activity such as that of a virus or a caspase.

The disclosure provides, in one aspect, a vector comprising a nucleic acid comprising a nucleotide sequence comprising a 5′ untranslated region, a nucleotide sequence encoding a degron, a nucleotide sequence encoding a cleavage site, and a nucleotide sequence encoding a reporter protein. In some embodiments, the nucleotide sequence encoding the degron is positioned 3′ to the nucleotide sequence encoding the 5′ untranslated region, the nucleotide sequence encoding the cleavage site is positioned 3′ to the nucleotide sequence encoding the degron, and the nucleotide sequence encoding the reporter protein is positioned 3′ to the nucleotide sequence encoding the cleavage site. In some embodiments, the nucleotide sequence encoding the reporter protein is positioned 3′ to the nucleotide sequence encoding the 5′ untranslated region, the nucleotide sequence encoding the cleavage site is positioned 3′ to the nucleotide sequence encoding the reporter protein, and the nucleotide sequence encoding the degron is positioned 3′ to the nucleotide sequence encoding the cleavage site. In some embodiments, the 5′ untranslated region comprises a 5′ untranslated region of the SARS-CoV-2 virus genome. In some embodiments, the degron comprises a ubiquitin domain. In some embodiments, the ubiquitin domain comprises the amino acid sequence of SEQ ID NO: 4. In some embodiments, the cleavage site is specifically cleaved by 3C-like protease. In some embodiments, the cleavage site comprises the amino acid sequence of SEQ ID NO: 6. In some embodiments, the vector comprises a nucleotide sequence that is at least 75%, 80%, 85%, 90%, 95%, 96%, 97%, 98%, 99%, or 100% identical to a nucleotide sequence selected from the group consisting of SEQ ID NOs: 7-9. In some embodiments, the vector comprises the nucleotide sequence of SEQ ID NO: 7. In some embodiments, the vector comprises the nucleotide sequence of SEQ ID NO: 8. In some embodiments, the vector comprises the nucleotide sequence of SEQ ID NO: 9. In some embodiments, the cleavage site is specifically cleaved by papain-like protease. In some embodiments, the cleavage site is specifically cleaved by a caspase. In some embodiments, the reporter comprises a fluorescent protein. In some embodiments, the fluorescent protein comprises mNeonGreen. In some embodiments, the fluorescent protein comprises Red Fluorescent Protein. In some embodiments, the nucleotide sequence comprising the 5′ untranslated region comprises a nucleotide sequence that is at least 75%, 80%, 85%, 90%, 95%, 96%, 97%, 98%, 99% or 100% identical to nucleotides 1,613-1,877 of SEQ ID NO: 11; the degron comprises an amino acid sequence that has 0, 1, 2, 3, 4 or 5 amino acid changes compared to the amino acid sequence of SEQ ID NO: 4; and the cleavage site comprises an amino acid sequence that has 0, 1, 2, 3, 4 or 5 amino acid changes compared to the amino acid sequence of SEQ ID NO: 6. In some embodiments, the reporter protein comprises an amino acid sequence that is at least 75%, 80%, 85%, 90%, 95%, 96%, 97%, 98%, 99%, or 100% identical to SEQ ID NO: 13 or 14. In some embodiments, the vector is packaged in a baculovirus. In some embodiments, the baculovirus is BacMam. In some embodiments, the vector comprises a nucleic acid comprising the sequence of positions 1614 to 2208 of any one of SEQ ID NO: 7, SEQ ID NO: 8, and SEQ ID NO: 9. In some embodiments, the vector comprises a nucleic acid that comprises at least 75%, 80%, 85%, 90%, 95%, 96%, 97%, 98%, 99%, or 100% identity to the sequence of SEQ ID NO: 23. In some embodiments, the vector comprises a nucleic acid that comprises at least 75%, 80%, 85%, 90%, 95%, 96%, 97%, 98%, 99%, or 100% identity to the sequence of SEQ ID NO: 24. In some embodiments, the vector encodes an amino acid sequence comprising at least 75%, 80%, 85%, 90%, 95%, 96%, 97%, 98%, 99%, or 100% identity to the sequence of SEQ ID NO: 25.

In another aspect, the present disclosure provides a vector comprising a nucleic acid comprising a nucleotide sequence encoding a 5′ untranslated region; a nucleotide sequence encoding a degron; a nucleotide sequence encoding a cleavage site; a nucleotide sequence encoding a first reporter protein; and a nucleotide sequence encoding a second reporter protein. In some embodiments, the nucleotide sequence encoding the degron is positioned 3′ to the nucleotide sequence encoding the 5′ untranslated region, the nucleotide sequence encoding the cleavage site is positioned 3′ to the nucleotide sequence encoding the degron, and the nucleotide sequence encoding the reporter protein is positioned 3′ to the nucleotide sequence encoding the cleavage site. In some embodiments, the nucleotide sequence encoding the first reporter protein is positioned 3′ to the nucleotide sequence encoding the 5′ untranslated region, the nucleotide sequence encoding the cleavage site is positioned 3′ to the nucleotide sequence encoding the first reporter protein, and the nucleotide sequence encoding the degron is positioned 3′ to the nucleotide sequence encoding the cleavage site. In some embodiments, the 5′ untranslated region comprises a 5′ untranslated region of the SARS-CoV-2 virus genome. In some embodiments, the degron comprises a ubiquitin domain. In some embodiments, the ubiquitin domain comprises the amino acid sequence of SEQ ID NO: 4. In some embodiments, the cleavage site is specifically cleaved by 3C-like protease. In some embodiments, the cleavage site comprises the amino acid sequence of SEQ ID NO: 6. In some embodiments, the vector comprises a nucleotide sequence that is at least 75%, 80%, 85%, 90%, 95%, 96%, 97%, 98%, 99%, or 100% identical to a of the nucleotide sequence of SEQ ID NO: 11. In some embodiments, the cleavage site is specifically cleaved by papain-like protease. In some embodiments, the cleavage site is specifically cleaved by a caspase. In some embodiments, both the first reporter protein and the second reporter protein each comprise a fluorescent protein. In some embodiments, the first reporter protein comprises mNeonGreen, and the second reporter protein comprises Red Florescent Protein. In some embodiments, the second reporter protein comprises mNeonGreen, and the first reporter protein comprises Red Florescent Protein. In some embodiments, the vector further comprises a self-cleaving peptide encoded by nucleotides that are positioned between the nucleotides encoding the first reporter protein and the nucleotides encoding second reporter protein. In some embodiments, the vector further comprises a self-cleaving peptide encoded by nucleotides that are positioned between the nucleotides encoding the degron and the nucleotides encoding second reporter protein. In some embodiments, the self-cleaving peptide, if completely translated, would comprise the amino acid sequence of SEQ ID NO: 15. In some embodiments, wherein the nucleotide sequence comprising the 5′ untranslated region comprises a nucleotide sequence that is at least 75%, 80%, 85%, 90%, 95%, 96%, 97%, 98%, 99% or 100% identical to nucleotides 1,613-1,877 of SEQ ID NO: 11; the degron comprises an amino acid sequence that has 0, 1, 2, 3, 4 or 5 amino acid changes compared to the amino acid sequence of SEQ ID NO: 4; and the cleavage site comprises an amino acid sequence that has 0, 1, 2, 3, 4 or 5 amino acid changes compared to the amino acid sequence of SEQ ID NO: 6. In some embodiments, the first reporter protein and the second reporter protein each comprise an amino acid sequence that is at least 75%, 80%, 85%, 90%, 95%, 96%, 97%, 98%, 99%, or 100% identical to SEQ ID NO: 13 or 14. In some embodiments, the vector is packaged in a baculovirus. In some embodiments, the baculovirus is BacMam. In some embodiments, the vector comprises a nucleic acid that comprises at least 75%, 80%, 85%, 90%, 95%, 96%, 97%, 98%, 99%, or 100% identity to the sequence of SEQ ID NO: 23. In some embodiments, the vector comprises a nucleic acid that comprises at least 75%, 80%, 85%, 90%, 95%, 96%, 97%, 98%, 99%, or 100% identity to the sequence of SEQ ID NO: 24. In some embodiments, the vector encodes an amino acid sequence comprising at least 75%, 80%, 85%, 90%, 95%, 96%, 97%, 98%, 99%, or 100% identity to the sequence of SEQ ID NO: 25. In some embodiments, the vector comprises a nucleic acid comprising the sequence of positions 1614 to 2208 of any one of SEQ ID NO: 7, SEQ ID NO: 8, and SEQ ID NO: 9.

In another aspect, the disclosure provides a biosensor encoded by a vector comprising a 5′ untranslated region, a degron positioned 3′ to the untranslated region, a cleavage site positioned 3′ to the degron, a sequence encoding a first reporter protein positioned 3′ to the cleavage site, and, optionally, a sequence encoding a second reporter protein positioned 3′ to the first reporter protein.

In yet another aspect, the disclosure provides a cell comprising a vector comprising a 5′ untranslated region, a degron positioned 3′ to the untranslated region, a cleavage site positioned 3′ to the degron, a sequence encoding a first reporter protein positioned 3′ to the cleavage site, and, optionally, a sequence encoding a second reporter protein positioned 3′ to the first reporter protein.

The disclosure provides, in one aspect, a cell comprising a biosensor encoded by a vector comprising a 5′ untranslated region, a degron positioned 3′ to the untranslated region, a cleavage site positioned 3′ to the degron, a sequence encoding a first reporter protein positioned 3′ to the cleavage site, and, optionally, a sequence encoding a second reporter protein positioned 3′ to the first reporter protein.

In another aspect, the disclosure provides a method for detecting protease activity in a cell comprising measuring a signal from a biosensor encoded by a vector comprising a 5′ untranslated region, a degron positioned 3′ to the untranslated region, a cleavage site positioned 3′ to the degron, a sequence encoding a first reporter protein positioned 3′ to the cleavage site, and, optionally, a sequence encoding a second reporter protein positioned 3′ to the first reporter protein.

In another aspect, the disclosure provides a method for detecting protease activity in a cell comprising measuring a signal from at least two reporter proteins, both encoded by a vector comprising a 5′ untranslated region, a degron positioned 3′ to the untranslated region, a cleavage site positioned 3′ to the degron, a sequence encoding a first reporter protein positioned 3′ to the cleavage site, and, optionally, a sequence encoding a second reporter protein positioned 3′ to the first reporter protein.

In yet another aspect, the disclosure provides a method of detecting SARS-CoV-2 infection in a sample from a subject, wherein the sample comprises cells from the subject, comprising introducing an effective amount of a vector comprising a 5′ untranslated region, a degron positioned 3′ to the untranslated region, a cleavage site positioned 3′ to the degron, a sequence encoding a first reporter protein positioned 3′ to the cleavage site, and, optionally, a sequence encoding a second reporter protein positioned 3′ to the first reporter protein, to the cells in the sample and measuring a signal from the reporter. In some embodiments, the method further comprises measuring at least two signals from the reporter.

In another aspect, the disclosure provides a method of detecting a protease inhibitor specific for a protease present in a cell comprising introducing an effective amount of a vector comprising a 5′ untranslated region, a degron positioned 3′ to the untranslated region, a cleavage site positioned 3′ to the degron, a sequence encoding a first reporter protein positioned 3′ to the cleavage site, and, optionally, a sequence encoding a second reporter protein positioned 3′ to the first reporter protein to the cell and measuring a signal from the reporter. In some embodiments, the protease is introduced to the cell with a vector. In some embodiments, the vector is packaged in a baculovirus. In some embodiments, the baculovirus is BacMam. In some embodiments, the method further comprises measuring at least two signals from the reporter.

In still another aspect, the disclosure provides a method of measuring replication of a virus in a cell comprising introducing an effective amount of a vector comprising a 5′ untranslated region, a degron positioned 3′ to the untranslated region, a cleavage site positioned 3′ to the degron, a sequence encoding a first reporter protein positioned 3′ to the cleavage site, and, optionally, a sequence encoding a second reporter protein positioned 3′ to the first reporter protein, to the cell and measuring a signal from the reporter. In some embodiments, the method further comprises measuring at least two signals from the reporter.

### BRIEF DESCRIPTION OF THE DRAWINGS

Aspects, features, benefits, and advantages of the embodiments described herein will be apparent with regard to the following description, appended claims, and accompanying drawings where:

FIG. 1 depicts certain sequence features of the 3CL protease biosensor. The nucleotide sequence shown is SEQ ID NO: 16, and the protein sequence shown is SEQ ID NO: 17. These features include a ubiquitin domain at the N terminus of the protein, wherein cleavage of the ubiquitin should leave an arginine at the new N-terminus. Following the ubiquitin domain is the 3CL protease cleavage site comprising 33 amino acids found between NSP9 (non-structural protein 9) and NSP10 (nonstructural protein 10) in SARS-CoV-2. Finally, there is a mNeonGreen fluorescent reporter protein following the 3CL cleavage site.

FIG. 2 depicts a construct used to express 3CL protease. The open reading frame for this protease has an additional Kozak translation start and ATG at the 5′ end of the coding region to translate the protein.

FIG. 3A-3C depict three versions of a construct or vector encoding the 3CL protease biosensor. FIG. 3A depicts a construct or vector encoding a biosensor comprising a “slow” degradation rate and comprising N-terminal E. This construct or vector comprises the nucleotide sequence of SEQ ID NO: 7. FIG. 3B depicts a construct or vector encoding a biosensor comprising a “medium” degradation rate and comprising N-terminal Y. This biosensor construct or vector comprises the nucleotide sequence of SEQ ID NO: 8. FIG. 3C depicts a construct or vector encoding a biosensor comprising a “fast” degradation rate and comprising N-terminal R. This construct or vector comprises the nucleotide sequence of SEQ ID NO: 9.

FIGS. 4A-4C depict a comparative assay of the fluorescence detected in the prototype biosensors of Example 1 designed to have either “fast” (encoding N-terminal R), “medium” (encoding N-terminal A), or “slow” (encoding N-terminal E) degradation rates. FIG. 4A depicts a schematic of the sequence features of all biosensors tested. FIG. 4B graphically depicts the fluorescence detected for all three biosensors. All three prototype biosensors showed fluorescence in the presence of 3CL protease but little fluorescence in the absence of 3CL protease. The strongest fluorescent signal was detected for the “fast” biosensor. FIG. 4C depicts a comparison in fluorescence detected in samples wherein 3CL protease is added and negative control samples without 3CL protease.

FIG. 5 depicts an optimization assay of BacMam viral delivery of 3CL protease and 3CL protease biosensors. Both of the tested biosensors, the fast and medium rate biosensors, reported the presence of 3CL protease activity in a dose dependent manner. The fast biosensor showed a steeper fluorescence/3CL protease dependence, indicating that it is the most sensitive of the biosensors to protease activity levels.

FIG. 6 depicts a live cell assay using the 3CL protease “fast” biosensor to determine if GC376 (Anivive Lifesciences) inhibits SARS-CoV-2 3CL protease enzyme activity. In this assay, a dose dependent inhibition of the 3CL protease activity by GC376 was observed.

FIG. 7 depicts a live cell assay of the dose dependent inhibition of the 3CL protease activity by GC376 wherein the amount of 3CL protease biosensor was varied by including 5 μl, 7.5 μl, and 10 μl of a BacMam vector suspension encoding the biosensor.

FIG. 8 depicts fluorescent images of HEK293 cells incubated overnight with either 100 nM or 31.6 μM GC376. The fluorescent signal is produced by the 3CL protease biosensor. The fluorescence visible from the cells incubated with 31.6 μM GC376 is much lower from the cells incubated with 100 nM GC376.

FIG. 9 depicts an exemplary embodiment of a biosensor according to the present disclosure which comprises two reporter proteins.

FIGS. 10A-10B depict fluorescent images of HEK293 cells in the red (bottom panels) and green (top panels) emission channels that were transduced with a BacMam viral vector encoding the biosensor of Example 5. FIG. 10A shows cells that were not co-transduced with the 3CL protease. FIG. 10B shows cells that were co-transduced with the 3CL protease.

FIGS. 11A-11B depict fluorescence levels of HEK293 cells co-transduced with the biosensor of Example 5 and the 3CLpro protease. Cells were treated with varying amounts of GC376 protease inhibitor. FIG. 11A shows fluorescence data for the green channel. FIG. 11B shows fluorescence data for the red channel.

FIGS. 12A-12C depict fluorescence levels or fluorescence ratios of HEK293 cells co-transduced with BacMam viral vectors expressing the biosensor of Example 5 and the 3CL protease. FIG. 12A shows fluorescence data for the green channel three concentrations of GC376 and three replicates of each. FIG. 12B shows fluorescence data for the green channel vs the concentration of GC376. FIG. 12C shows fluorescence data for the green channel normalized to fluorescence in the red channel vs the concentration of GC376.

FIGS. 13A-13B depict fluorescence ratios of HEK293 cells co-transduced with the biosensor of Example 5 and the 3CL protease. FIG. 13A shows fluorescence data for cells that were treated with compound 43 at the noted concentration and treated or untreated with CP100356. FIG. 13B shows fluorescence data for cells that were treated with GC376 at the noted concentration and treated or untreated with CP100356.

FIGS. 14A-14B depict fluorescent images of Vero cells in the green channel that have been transduced at the indicated multiplicity of infection (MOI). FIG. 14A shows fluorescent images of Vero cells co-transduced with the biosensor of Example 5 and SARS-CoV-2 virus.

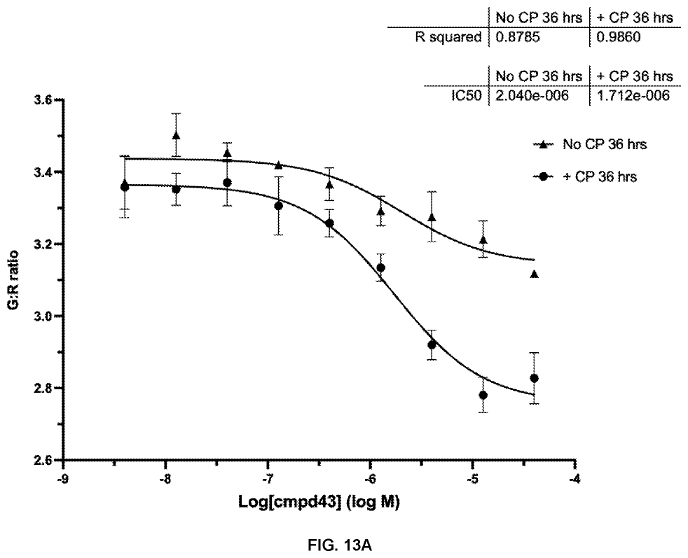

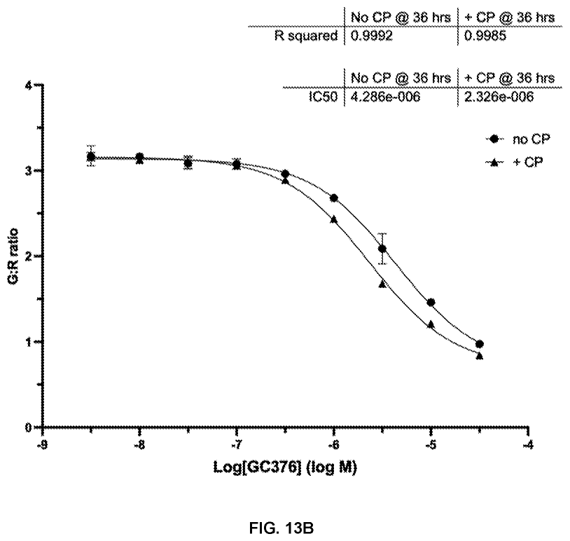

FIG. 13B shows fluorescent images of Vero cells transduced with the icSARS-CoV-2 mNG virus.

### DETAILED DESCRIPTION

It will be appreciated that for clarity, the following disclosure will describe various aspects of embodiments. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting. The section headings used herein are for organizational purposes only and are not to be construed as limiting the subject matter described.

The term “identical” or “percent identical” with reference to a nucleotide sequence or an amino acid sequence refers to at least two nucleotide or at least two amino acid sequences or subsequences that have a specified percentage of nucleotides or amino acids, respectively, that are the same, when compared and aligned for maximum correspondence, as measured using a sequence comparison algorithm or by visual inspection. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information.

The term “nucleotide sequence” as used herein refers to DNA and RNA nucleotide sequences. In some embodiments, vectors used herein are made up of DNA nucleotide sequences.

The novel coronavirus severe acute respiratory syndrome coronavirus 2 (SARS-CoV-2) hijacks the human ACE2 protein as a receptor to enter cells, causing severe respiratory diseases. In some embodiments, biosensors effective in detecting SARS-CoV-2 are disclosed. In some embodiments, cellular assays to detect compounds capable for inhibiting the replication of SARS-CoV-2 are disclosed.

As used herein a “biosensor” is one or more recombinant proteins that is/are capable of producing a signal, via a reporter, in response to (1) a viral infection and/or (2) the activity of a protease. The signal can be easily interpretable, such as that from one or more light-emitting reporter proteins (e.g., fluorescent or luminescent proteins).

As used herein a “vector” refers to a recombinant nucleic acid construct that encodes at least one transcript capable of being expressed in a cell. A vector can be, for example, a nucleic acid itself (such as a plasmid or bacmid) or a viral vector whose genome comprises the vector sequence. A vector can encode a biosensor as disclosed herein.

As used herein, “coronavirus(es)” (CoVs) are members of the family Coronaviridae of the Nidovirales order. Coronaviruses can be further subdivided into four groups, the alpha, beta, gamma and delta coronaviruses. However, the viruses were initially sorted into these groups based on serology but are now divided by phylogenetic clustering (Fehr et al., Methods Mol Biol. 2015; 1282: 1-23).

In some embodiments, a coronavirus detected by the biosensor of the present disclosure can be an alphacoronavirus, e.g., human coronavirus 229E (HCoV-229E), porcine epidemic diarrhea virus (PEDV), human coronavirus NL63 (HCoV-NL63), or alphacoronavirus 1. In some embodiments, a coronavirus detected by the biosensor of the present disclosure can be a betacoronavirus, e.g., betacoronavirus 1, human coronavirus 0C43 (HCoV-0C43), severe acute respiratory syndrome coronavirus (SARS-CoV), human coronavirus HKU1 (HCoV-HKU1), Middle East respiratory syndrome-related coronavirus (MERS-CoV), or severe acute respiratory syndrome coronavirus 2 (SARS-CoV-2). In some embodiments, a coronavirus detected by the biosensor of the present disclosure can be a gammacoronavirus. In some embodiments, a coronavirus detected by the biosensor of the present disclosure can be a deltacoronavirus.

Seven strains of human coronaviruses are known: human coronavirus 229E (HCoV-229E); human coronavirus 0C43 (HCoV-0C43); severe acute respiratory syndrome coronavirus (SARS-CoV); human coronavirus NL63 (HCoV-NL63, New Haven coronavirus); human coronavirus HKU1; middle East respiratory syndrome-related coronavirus (MERS-CoV, previously known as novel coronavirus 2012 and HCoV-EMC); and SARS-CoV-2, previously known as 2019-nCoV or “novel coronavirus 2019”.

Coronavirus disease 2019 (COVID-19) is an infectious disease caused by SARS-CoV-2. Common symptoms include fever, cough and shortness of breath. Muscle pain, sputum production and sore throat are less common. While the majority of cases result in mild symptoms, some progress to severe pneumonia and multi-organ failure. The rate of deaths per number of diagnosed cases is on average 3.4%, ranging from 0.2% in those less than 20 to approximately 15% in those over 80 years old.

Coronaviruses are enveloped, non-segmented positive-sense RNA viruses. They contain approximately 30 kilobase (kb) genomes. Other features of coronaviruses include: i) a highly conserved genomic organization, with a large replicase gene preceding structural and accessory genes; ii) expression of many nonstructural genes by ribosomal frameshifting; iii) several unique or unusual enzymatic activities encoded within the large replicase-transcriptase polyprotein; and iv) expression of downstream genes by synthesis of 3′ nested sub-genomic mRNAs.

3C-like protease (3CL protease) and papain-like protease (PL protease) are essential for replication of coronaviruses. The coronavirus genome contains two overlapping open reading frames that encode polyproteins pp 1a and pp 1b. Both 3CL protease and PL protease function together to cleave these polyproteins to form 16 mature proteins (Rathnayake et al. Science Translational Medicine 19 Aug. 2020: Vol. 12, Issue 557.) For this reason, both CL protease and PL protease are attractive targets for inhibitors of coronaviruses. 3CL protease inhibitors have been shown to block MERS-CoV and SARS-CoV-2 coronavirus replication in vitro and improve survival in MERS-CoV-infected mice (Rathnayake et al. Science Translational Medicine 19 Aug. 2020: Vol. 12, Issue 557.).

### Fluorescent Biosensor

Many biological processes are not easily monitored or visualized. Accordingly, the present disclosure provides a fluorescent biosensor that is capable of detecting the activity of a certain proteases which may or not be present a cell. As long as the cell comprises the biosensor, the presence or absence of detectable protease activity in the cell can be determined. The protease can be any protease that produces substrate protein cleavage in response to the presence of known, specific amino acid sequence in the substrate.

In some embodiments, the biosensor detects activity of a viral protease. In some embodiments, the virus is a coronavirus. In some embodiments, the virus is a human coronavirus. In some embodiments, the virus is HCoV-229E. In some embodiments, the virus is HCoV-0C43. In some embodiments, the virus is SARS-CoV. In some embodiments, the virus is HCoV-NL63. In some embodiments, the virus is human coronavirus HKU1. In some embodiments, the virus is MERS-CoV. In some embodiments, the virus is SARS-CoV-2. In some embodiments, the protease is the coronavirus 3CL protease or the PL protease.

SARS-CoV-2 can only be safely handled in Biosafety Level 3 laboratories. The virus can be readily propagated in a variety of human and primate cell lines, including Vero E6 cells (ATCC) and Calu 3 cells)(ATCC®). However, it can be difficult to identify infected cells until the cytopathic effects of the virus are obvious. An alternative is to fix the cells and then process them with antibodies directed against one of the viral proteins, a process that is time consuming and involves killing the cells with fixative and permeabilizing them with detergents so that the antibodies can penetrate the cells.

In some embodiments, the biosensor detects activity of a mammalian protease. In some embodiments, the biosensor detects activity of a caspase protease. In some embodiments, the biosensor functions as an apoptosis biosensor. Exemplary caspases, along with the peptide sequence they cleave are listed below. (See Julien, 0., and Wells, J.A. (2017). Caspases and their substrates. Cell Death Differ. 24, 1380-1389, which is incorporated by reference herein, in its entirety). Each peptide sequence can be included as a cleavage site in a biosensor of the present disclosure to detect activity of the corresponding caspase.

TABLE 1
Caspases and corresponding cleavage site
sequences

| Caspase protein | Cleavage site sequence |
|---|---|
| Caspase 3 | DVED (SEQ ID NO: 18) |
| Caspase 1 | WEHD (SEQ ID NO: 19) |
| Caspase 2 | VDVAD (SEQ ID NO: 20) |
| Caspase 4 | LEVD (SEQ ID NO: 21) |
| Caspase 8 | LETD (SEQ ID NO: 22) |

In some embodiments, a simple, easy to use fluorescent biosensor that will rapidly report protease activity (e.g., that of virus or that of apoptosis) in living cells with no additional reagents, cell fixation, or antibodies is disclosed. In some embodiments, the biosensor is engineered to have a degron (ubiquitin domain) that ensures the half-life of the fusion protein is too short to produce a detectable signal. Some embodiments include a protease cleavage site that is positioned in between the degron and a reporter fluorescent protein. Cleavage separates the degron from the reporter such that the reporter half-life increases and the reporter can produce a detectable signal.

In some embodiments, the biosensor includes or encodes more than one fluorescent protein such as two, three, or more fluorescent proteins. In some embodiments, the biosensor includes or encodes two fluorescent proteins—a first which produces a detectable signal that is dependent on protease cleavage and a second which is expressed and detectable independent of protease activity.

### 5′ Untranslated Region

NSP1 (non-structural protein 1), of the SARS-CoV virus serves to block host mRNA translation. The viral transcripts, however, evade this nuclease because each transcript carries the 5′ UTR of the virus which forms a step loop structure presumably recognized by the NSP1 (Tanaka, et al. 2012. “Severe Acute Respiratory Syndrome Coronavirus nspl Facilitates Efficient Propagation in Cells through a Specific Translational Shutoff of Host mRNA.” Journal of Virology 86 (20): 11128-37).

In some embodiments, the biosensor includes a 5′ untranslated (UTR). In some embodiments, the 5′ UTR comprises genomic DNA from the organism of interest. In some embodiments, the 5′ UTR comprises virus genome DNA. In some embodiments, the 5′ UTR comprises DNA from the SARS-CoV-2 virus genome. In some embodiments, the 5′ UTR is transcribed and protects the mRNA from viral proteins that destroy host mRNAs. In some embodiments, the 5′ UTR is transcribed and protects the mRNA from NSP1 nuclease. In some embodiments, the presence of the 5′ UTR allows the biosensor to be used in cells carrying live virus.

In some embodiments, the 5′ UTR is encoded by the nucleotide sequence of nucleotides 1,613-1,877 of SEQ ID NO: 11. In some embodiments, the 5′ UTR is encoded by a nucleotide sequence that is at least 75%, 80%, 85%, 90%, 95%, 96%, 97%, 98%, 99% or 100% identical to nucleotides 1,613-1,877 of SEQ ID NO: 11.

Any suitable 5′ UTR can be employed when the biosensor is designed to detect coronavirus protease activity. For example, a biosensor with a human coronavirus protease cleavage site can be engineered to contain the corresponding human coronavirus 5′ UTR.

### Degron

The term “degron” is used to refer to a degradation sequence. In some embodiments, the presence of a degron in the biosensor reduces the half-life of a protein by targeting the protein for degradation via ubiquitination. In some embodiments the degron is encoded by a nucleic acid comprising a nucleotide sequence comprising SEQ ID NO: 3. In some embodiments the degron is encoded by a nucleic acid comprising a nucleotide sequence that is at least 75%, 80%, 85%, 90%, 95%, 96%, 97%, 98%, 99%, or 100% identical to the nucleotide sequence SEQ ID NO: 3. In some embodiments, the translated ubiquitin domain comprises the amino acid sequence of SEQ ID NO: 4. In some embodiments, the translated ubiquitin domain comprises an amino acid sequence that has 1, 2, 3, 4 or 5 amino acid changes compared to the amino acid sequence of SEQ ID NO: 4. In some embodiments, the degron is encoded by a nucleic acid that encodes an amino acid sequence comprising SEQ ID NO: 4. In some embodiments, the degron is encoded by a nucleic acid sequence that encodes an amino acid sequence comprising 1, 2, 3, 4 or 5 amino acid changes compared to SEQ ID NO: 4.

In some embodiments, the degron is comprised in the N-terminus of a translated protein. In some embodiments, placing the degron in the N-terminus of a biosensor shortens the half-life of the biosensor to a degree that it does not have enough time to fold and form a functional fluorophore.

In some embodiments, the degron is positioned 3′ to a UTR on a nucleic acid comprising a nucleotide sequence encoding a biosensor.

Any type of destabilizing motif can be used to shorten the half-life of the protein. In some embodiments, the destabilizing motif is ubiquitin-independent. In some embodiments, the destabilizing motif is ubiquitin-dependent. In some embodiments, a PEST sequence can serve as a degron. In some embodiments, the vector encoding the biosensor comprises a nucleic acid comprising components in the following order relative to each other: 5′-degron-cleavage site-reporter protein-3′. In some embodiments, the vector encoding the biosensor comprises a nucleic acid comprising components in the following order relative to each other: 5′-reporter protein-cleavage site-degron-3′. In some embodiments, either of these two biosensors may comprise a nucleic acid comprising a second reporter protein. The nucleic acid comprising the second reporter protein may be located either 5′ or 3′ of the block of the other three components and maybe separated therefrom by a nucleic acid encoding a self-cleaving peptide.

### Protease Cleavage Site

A protease is an enzyme that catalyzes the breakdown of a protein into smaller polypeptide units. A protease cleavage site is an amino acid location where a protease interacts with a protein and breaks it into smaller polypeptide units. In some embodiments, the biosensor comprises a protease cleavage site. In some embodiments a protease cleavage site is positioned between a degron and a fluorescent protein. In these embodiments, if the protease corresponding to the protease cleavage site is present, the degron is cleaved from the fluorescent protein such that the half-life of the remaining reporter protein increases.

In some embodiments, the protease cleavage site is positioned C-terminal to a degron and N-terminal to a reporter protein of the biosensor. In some embodiments, the protease cleavage site is positioned N-terminal to a degron and C-terminal to a reporter protein of the biosensor.

In some embodiments, the protease cleavage site is cleaved by a viral protease. In some embodiments, the protease cleavage site is cleaved by a 3CL protease of a coronavirus. In some embodiments, the protease cleavage site is cleaved by a 3CL protease of a human coronavirus. In some embodiments, the protease cleavage site is cleaved by a 3CL protease of HCoV-229E. In some embodiments, the protease cleavage site is cleaved by a 3CL protease of HCoV-0C43. In some embodiments, the protease cleavage site is cleaved by a 3CL protease of SARS-CoV. In some embodiments, the protease cleavage site is cleaved by a 3CL protease of HCoV-NL63. In some embodiments, the protease cleavage site is cleaved by a 3CL protease of human coronavirus HKU1. In some embodiments, the protease cleavage site is cleaved by a 3CL protease of MERS-CoV. In some embodiments, the protease cleavage site is cleaved by a 3CL protease of SARS-CoV-2.

In some embodiments, the translated protease cleavage site is encoded by a nucleic acid comprising the nucleotide sequence of SEQ ID NO: 5. In some embodiments, the translated protease cleavage site is cleaved by 3CL protease of SARS-CoV-2 and is encoded by a nucleic acid comprising a nucleotide sequence that has 1, 2, 3, 4, 5, 6, 7, 8, 9 or 10 nucleotide changes compared to the nucleotide sequence of SEQ ID NO: 5. In some embodiments, the translated protease cleavage site is cleaved by a 3CL protease of SARS-CoV-2 and comprises the amino acid sequence of SEQ ID NO: 6. In some embodiments, the translated protease cleavage site is cleaved by 3CL protease of SARS-CoV-2 and comprises an amino acid sequence that has 1, 2, 3, 4 or 5 amino acid changes compared to the amino acid sequence of SEQ ID NO: 6, but is still capable of being specifically cleaved by 3CL protease.

The disclosure also provides nucleic acids that encode the amino acid sequence of SEQ ID NO: 6. In some embodiments, the nucleic acids encodes an amino acid sequence comprising 1, 2, 3, 4 or 5 amino acid changes compared to the amino acid sequence of SEQ ID NO: 6.

Immediately before and after the protease cleavage site, additional amino acid residues may be placed. For example, nucleotides 2,109-2,141 and 2,172-2,207 of SEQ ID NO: 11 encode amino acids which are part of neither the degron nor the mNeonGreen protein. These “buffer residues” function to provide additional steric clearance and/or flexibility for the protease to contact the substrate and promote the effective cleavage of the biosensor at or near the protease site. These buffer residues can comprise any amino acids. In some embodiments, residues which do not interfere with protein function (e.g., fluorescence) can be selected.

In some embodiments, the 3CL protease is encoded by a nucleotide sequence comprising SEQ ID NO: 1. In some embodiments, the 3CL protease is encoded by a nucleic acid comprising a nucleotide sequence that is at least 75%, 80%, 85%, 90%, 95%, or 100% identical to the nucleotide sequence of SEQ ID NO: 1.

In some embodiments, the 3CL protease comprises the amino acid sequence of SEQ ID NO: 10. In some embodiments, the 3CL protease comprises an amino acid sequence that is at least 75%, 80%, 85%, 90%, 95%, or 100% identical to the amino acid sequence of SEQ ID NO: 10.

The disclosure also provides nucleic acids that encode the amino acid sequence of SEQ ID NO: 10. In some embodiments, the nucleic acids encode an amino acid sequence with at least 75%, 80%, 85%, 90%, 95%, or 100% identity to the amino acid sequence of SEQ ID NO: 10.

In some embodiments, the protease cleavage site is cleaved by a papain like (PL) protease of SARS-CoV-2. In some embodiments, the PL protease comprises the amino acid sequence of SEQ ID NO: 12. In some embodiments, the PL protease comprises an amino acid sequence that is at least 75%, 80%, 85%, 90%, 95%, or 100% identical to the amino acid sequence of SEQ ID NO: 12.

The disclosure also provides nucleic acids that encode the amino acid sequence of SEQ ID NO: 12. In some embodiments, the nucleic acids encode an amino acid sequence with at least 75%, 80%, 85%, 90%, 95%, or 100% identity to the amino acid sequence of SEQ ID NO: 12.

It is noted that SEQ ID NOs 1, 10, and 12 do not include an N-terminal methionine or a codon therefor. The native coronavirus sequence does not contain these methionine residues since these proteases are initially translated as a single pro-protein and then proteolytically processed by the PL protease and 3CL protease. However, it is understood that when these protease sequences are expressed recombinantly, separately from the balance of the coronavirus genome, a start codon (and therefore an N-terminal methionine) may be employed.

In some embodiments, the protease cleavage site is cleaved by a mammalian protease. In some embodiments, the protease cleavage site is cleaved by a caspase. In some embodiments, the protease cleavage site is cleaved by a caspase and the biosensor is an apoptosis biosensor.

### Reporter Protein

In some embodiments, the biosensor includes a reporter protein. In some embodiments, the reporter protein is positioned 3′ to a protease cleavage site.

In some embodiments, the biosensor includes or encodes more than one fluorescent reporter protein such as two, three, or more fluorescent reporter proteins. In some embodiments, the biosensor includes or encodes two fluorescent proteins. In some embodiments, the first fluorescent protein can produce a detectable signal that is dependent on protease cleavage. In some embodiments, the second fluorescent protein can be expressed and detectable independent of protease activity.

For example, the first protein can provide a signal when the virus (which can supply the protease) is present in an infected cell. The second protein can provide a signal regardless of whether or not a host cell in infected, based only on whether or not the cell is healthy enough to express the second protein.

In some embodiments, lack of the signal from the first protein and the second protein can be due to either an unhealthy or dead cell. In some embodiments, lack of the signal from the first protein, but presence of signal from the second protein can be due to lack of the viral protease.

In some embodiments, the two or more fluorescent proteins produce fluorescent signals which are easily distinguishable from each other such as, for example, any two or more of blue/UV proteins, cyan proteins, green proteins, yellow proteins, orange proteins, red proteins, far-red proteins, near-infrared proteins, long stokes shift proteins, photoactivatible proteins, photoconvertible proteins, photoswitchable proteins, and luciferase.

In some embodiments, the two fluorescent reporter proteins comprise a green protein and a red protein.

A variety of reporter proteins can be used in the biosensor, including any suitable to provide a detectable, and optionally distinguishable, signal. In some embodiments, the reporter protein is a fluorescent protein. A fluorescent protein reporter protein is any protein that emits a fluorescent signal when activated by light or other electromagnetic radiation. In some embodiments the fluorescent protein is selected from the group consisting of blue/UV proteins (such as BFP, TagBFP, mTagBFP2, Azurite, EBFP2, mKalamal, Sirius, Sapphire, and T-Sapphire); cyan proteins (such as CFP, eCFP, Cerulean, SCFP3A, mTurquoise, mTurquoise2, monomeric Midoriishi-Cyan, TagCFP, and mTFP1); green proteins (such as: GFP, eGFP, meGFP (A208K mutation), Emerald, Superfolder GFP, Monomeric Azami Green, TagGFP2, mUKG, mWasabi, Clover, and mNeonGreen); yellow proteins (such as YFP, eYFP, Citrine, Venus, SYFP2, and TagYFP); orange proteins (such as Monomeric Kusabira-Orange, mKOK, mKO2, mOrange, and mOrange2); red proteins (such as RFP, mRaspberry, mCherry, mStrawberry, mTangerine, tdTomato, TagRFP, TagRFP-T, mApple, mRuby, and mRuby2); far-red proteins (such as mPlum, HcRed-Tandem, mKate2, mNeptune, and NirFP); near-infrared proteins (such as TagRFP657, IFP1.4, and iRFP); long stokes shift proteins (such as mKeima Red, LSS-mKatel, LSS-mKate2, and mBeRFP); photoactivatible proteins (such as PA-GFP, PAmCherryl, and PATagRFP); photoconvertible proteins (such as Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, and PSmOrange); and photoswitchable proteins (such as Dronpa). In some embodiments, the reporter protein has intrinsic fluorogenic or chromogenic activity (e.g., green, red, and yellow fluorescent bioluminescent proteins from a bioluminescent organism). In some embodiments, the reporter protein is a luciferase.

In some embodiments the biosensor comprises an mNeonGreen reporter protein encoded by a nucleic acid comprising nucleotides 2208-2915 of SEQ ID NO: 11. In some embodiments the biosensor comprises a reporter protein encoded by a nucleic acid that comprises a nucleotide sequence that is at least 75%, 80%, 85%, 90%, 95%, 96%, 97%, 98%, 99% or 100% identical to nucleotides 2208-2915 of SEQ ID NO: 11.

In some embodiments the biosensor comprises an mNeonGreen reporter protein comprising the amino acid sequence of SEQ ID NO: 13. In some embodiments the biosensor comprises a reporter protein comprising an amino acid sequence that is at least 75%, 80%, 85%, 90%, 95%, 96%, 97%, 98%, 99% or 100% identical to the amino acid sequence of SEQ ID NO: 13.

The disclosure also provides nucleic acids that encode the amino acid sequence of SEQ ID NO: 13. In some embodiments, the nucleic acids encode an amino acid sequence with at least 75%, 80%, 85%, 90%, 95%, or 100% identity to the amino acid sequence of SEQ ID NO: 13.

In some embodiments the biosensor comprises an RFP reporter protein encoded by a nucleic acid comprising nucleotides 2970-3758 of SEQ ID NO: 11. In some embodiments the biosensor comprises a reporter protein encoded by a nucleic acid that comprises a nucleotide sequence that is at least 75%, 80%, 85%, 90%, 95%, 96%, 97%, 98%, 99% or 100% identical to nucleotides 2970-3758 of SEQ ID NO: 11.

In some embodiments the biosensor comprises an RFP reporter protein comprising the amino acid sequence of SEQ ID NO: 14. In some embodiments the biosensor comprises a reporter protein comprising an amino acid sequence that comprises a nucleotide sequence that is at least 75%, 80%, 85%, 90%, 95%, 96%, 97%, 98%, 99% or 100% identical to the amino acid sequence of SEQ ID NO: 14.

The disclosure also provides nucleic acids that encode the amino acid sequence of SEQ ID NO: 14. In some embodiments, the nucleic acids encode an amino acid sequence with at least 75%, 80%, 85%, 90%, 95%, or 100% identity to the amino acid sequence of SEQ ID NO: 14.

In some embodiments, the biosensor comprises two unique reporter proteins such as fluorescent proteins that emit light at different wavelengths. In some embodiments, the biosensor comprises both the mNeonGreen and the RFP reporter proteins. The two reporter protein may be arranged with a self-cleaving peptide as described below.

In some embodiments, some or all of the reporter protein(s) can optionally comprise a nuclear localization signal (NLS). The NLS can be located N-terminal or C-terminal to the reporter protein. For example, the NLS can be located immediately N-terminal or C-terminal to the reporter protein. When the NLS is employed, the fluorescent signal can be beneficially localized to the nucleus of cells producing the signal. For example, counting fluorescent cells can be made easier when the cells' nuclei, but not cytosol regions, are fluorescent.

When employed, the NLS can comprise any peptide sequence that will result in transport of the reporter protein to the nucleus. In embodiments that employ two or more reporter proteins, some or all of reporter proteins can comprise an NLS. In some embodiments, the NLS can comprise an SV40 NLS. SEQ ID NO: 7 of the present disclosure comprises an SV40 NLS encoded immediately C-terminal to the mNeonGreen reporter protein coding sequence.

### Self-Cleaving Peptide

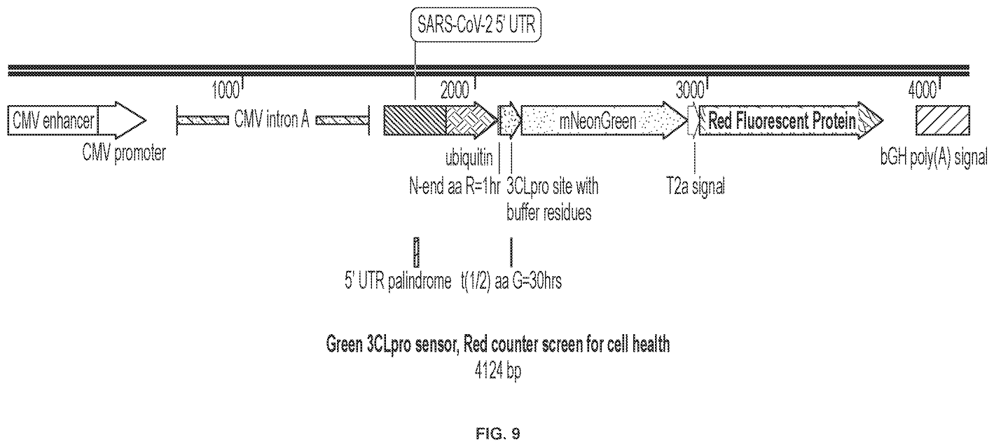

In embodiments where two reporter proteins are employed, a self-cleaving peptide sequence may be included in the biosensor. In some embodiments, the self-cleaving peptide sequence may be included between the sequences encoding the two reporter proteins of the biosensor. By including a self-cleaving peptide sequence in this manner, the first reporter protein can report on the activity (or lack thereof) of the protease, and the second reporter protein will be produced independently of the activity of the degron and protease. This allows the second reporter protein to report on efficiency of providing the vector to cells and the general health of the cells, while the first reporter protein will only accumulate if the protease activity to be detected is present. Overall, one example of a construct that encodes a self-cleaving peptide is provided by the nucleic acid shown in FIG. 9 and comprised in the nucleotide sequence of SEQ ID NO: 11. In these examples, the first reporter protein (mNeonGreen) is vulnerable to degradation via the ubiquitin degron unless the degron is removed by the 3CL protease. Additionally, a T2A peptide, followed by the RFP ORF, is present C-terminal to the mNeonGreen ORF. Thus the RFP will not be degraded since it is constitutively produced and separated from the ubiquitin degron.

In some embodiments, the self-cleaving peptide comprises a 2A peptide. The 2A peptide can induce ribosome skipping, which results in translation of separate polypeptides on either side of 2A peptide. In some embodiments, the self-cleaving peptide comprises a T2A peptide. In some embodiments, the T2A peptide is encoded by a nucleic acid comprising nucleotides 2,916-2,969 of SEQ ID NO: 11. In some embodiments, the T2A peptide is encoded by a nucleic acid comprising a nucleotide sequence that is at least 75%, 80%, 85%, 90%, 95%, 96%, 97%, 98%, 99% or 100% identical to nucleotides 2,916-2,969 of SEQ ID NO: 11. In some embodiments, the self-cleaving peptide comprises a P2A peptide. In some embodiments, the self-cleaving peptide comprises a E2A peptide. In some embodiments, the self-cleaving peptide comprises a F2A peptide.

The disclosure also provides nucleic acids that encode the amino acid sequence of SEQ ID NO: 15. In some embodiments, the nucleic acids encodes an amino acid sequence comprising 1, 2, 3, 4 or 5 amino acid changes compared to the amino acid sequence of SEQ ID NO: 15. The disclosure also provides nucleic acids that encode the self-cleaving peptides T2A, E2A or P2A.

### Biosensor

A biosensor of the present disclosure can be provided on a vector encoding the biosensor. The vector can comprise a nucleic acid comprising (1) a nucleotide sequence comprising a 5′ untranslated region, (2) a nucleotide sequence encoding a degron, (3) a nucleotide sequence encoding a cleavage site, and (4) a nucleotide sequence encoding a first reporter protein. These four components can be positioned relative to each other in a variety of configurations. However, it is beneficial for the three coding regions to be transcribed into a contiguous mRNA and translated such the cleavage site can direct protease-mediated cleavage that separates the degron from the first reporter protein.

In some embodiments, an “N-terminal degron” configuration is employed. In these embodiments, the nucleotide sequence encoding the degron is positioned 3′ to the nucleotide sequence encoding the 5′ untranslated region, the nucleotide sequence encoding the cleavage site is positioned 3′ to the nucleotide sequence encoding the degron, and the nucleotide sequence encoding the first reporter protein is positioned 3′ to the nucleotide sequence encoding the cleavage site.

In some embodiments, a “C-terminal degron” configuration is employed. In these embodiments, the nucleotide sequence encoding the first reporter protein is positioned 3′ to the nucleotide sequence encoding the 5′ untranslated region, the nucleotide sequence encoding the cleavage site is positioned 3′ to the nucleotide sequence encoding the first reporter protein, and the nucleotide sequence encoding the degron is positioned 3′ to the nucleotide sequence encoding the cleavage site. In some embodiments, no other nucleic acid elements intervene between the first reporter protein, the cleavage site, and the degron.

As described above and in some embodiments, a second reporter protein can be employed. When the second reporter protein is employed, the nucleic acid encoding the second reporter protein can be separated from other components of the biosensor (e.g., the nucleic acid encoding the first reporter or the nucleic acid encoding the degron) by a nucleic acid encoding a self-cleaving peptide, as described herein. Additionally, the second reporter protein can be encoded on the vector 3′ of the 5′ UTR. The nucleotide sequence encoding the second reporter protein can be 5′ or 3′ of the nucleotide sequence encoding a degron, the nucleotide sequence encoding the cleavage site, and the nucleotide sequence encoding the first reporter protein, with nucleotide sequence encoding the 2A site between the nucleotide sequence encoding the second reporter protein and the nucleotide sequence encoding a degron, the nucleotide sequence encoding the cleavage site, and the nucleotide sequence encoding the first reporter protein.

In some embodiments, a vector encoding the biosensor can comprise additional sequences that do no encode protein. In some embodiments, the vector can comprise a promoter suitable to drive expression of the biosensor. The promoter can comprise a promoter sufficiently strong to drive robust expression such as, for example a CMV promoter. Additionally, an enhancer such as a CMV enhancer can be employed to further increase expression. In some embodiments, the CMV enhancer can be encoded by positions 380-583 of SEQ ID NO: 11. In some embodiments, the CMV enhancer can be encoded by a nucleic acid comprising at least 75%, 80%, 85%, 90%, 95%, 96%, 97%, 98%, 99%, or 100% identity to positions 380-583 of SEQ ID NO: 11. In some embodiments, the CMV promoter can be encoded by positions 1-379 of SEQ ID NO: 11. In some embodiments, the CMV promoter can be encoded by a nucleic acid comprising at least 75%, 80%, 85%, 90%, 95%, 96%, 97%, 98%, 99%, or 100% identity to positions 1-379 of SEQ ID NO: 11.

In some embodiments, the vector can comprise an intron 5′ of the 5′ UTR. In some embodiments, the intron can comprise a CMV intron A. In some embodiments, the CMV intron A can be encoded by positions 719-1544 of SEQ ID NO: 11. In some embodiments, the CMV intron A can be encoded by a nucleic acid comprising at least 75%, 80%, 85%, 90%, 95%, 96%, 97%, 98%, 99%, or 100% identity to positions 719-1544 of SEQ ID NO: 11.

In some embodiments, the biosensor of the present disclosure comprises the amino acid sequence of SEQ ID NO: 25. In some embodiments, the biosensor of the present disclosure comprises an amino acid sequence at least 75%, 80%, 85%, 90%, 95%, 96%, 97%, 98%, 99%, or 100% identical to the amino acid sequence of SEQ ID NO: 25. The present disclosure also provides nucleic acids that encode the amino acid sequence of SEQ ID NO: 25. In some embodiments, the nucleic acids encode an amino acid sequence comprising at least 75%, 80%, 85%, 90%, 95%, 96%, 97%, 98%, 99%, or 100% identity to the amino acid sequence of SEQ ID NO: 25. SEQ ID NO: 25 comprises 2 groups of 12 amino acids on either side of the amino acids of the cleavage site (SEQ ID NO: 6). Both of these groups are optional and can be present or absent in the biosensor. These optional amino acids are shown with Xs in SEQ ID NO: 25 and, when present, can comprise any amino acids. These optional amino acids can be the buffer residues described herein. Examples of buffer residues can be found in the corresponding portions of SEQ ID NOs: 7-9 and 11. Buffer residues, when employed can comprise about 1-20 residues on either side of cleavage site.

In some embodiments, the biosensor of the present disclosure comprises the nucleic acid sequence of SEQ ID NO: 24. In some embodiments, the biosensor of the present disclosure comprises a nucleic acid sequence at least 75%, 80%, 85%, 90%, 95%, 96%, 97%, 98%, 99%, or 100% identical to the nucleic acid sequence of SEQ ID NO: 24. SEQ ID NO: 24 comprises 2 groups of 36 nucleotides on either side of the nucleotides encoding the cleavage site. Both of these groups are optional and can be present or absent in the biosensor. These optional nucleotides are shown with Ns in SEQ ID NO: 24 and, when present, can comprise any sense codons. These optional nucleotides can encode the buffer residues described herein. Examples of buffer residues can be found in the corresponding portions of SEQ ID NOs: 7-9 and 11.

In some embodiments, the biosensor of the present disclosure comprises the nucleic acid sequence of SEQ ID NO: 23. In some embodiments, the biosensor of the present disclosure comprises a nucleic acid sequence at least 75%, 80%, 85%, 90%, 95%, 96%, 97%, 98%, 99%, or 100% identical to the nucleic acid sequence of SEQ ID NO: 23. SEQ ID NO: 23 comprises 2 groups of 36 nucleotides on either side of the nucleotides encoding the cleavage site. Both of these groups are optional and can be present or absent in the biosensor. These optional nucleotides are shown with Ns in SEQ ID NO: 23 and, when present, can comprise any sense codons. These optional nucleotides can encode the buffer residues described herein. Examples of buffer residues can be found in the corresponding portions of SEQ ID NOs: 7-9 and 11.

### 3CL Protease Biosensor

3CL protease (also known as main protease (Mpro)) is encoded in by non-structural protein 5 (NSPS). In some embodiments, the 3CL protease is encoded by a nucleic acid that comprises the nucleotide sequence of SEQ ID NO: 1. In some embodiments, the sequence of 3CL protease comprises the amino acid sequence of SEQ ID NO: 10. In SARS-CoV-2, there are 13 different 3CL protease cleavage sites in the lab proprotein that are crucial to creating the suite of Nonstructural proteins (NSPs) involved in viral replication (Gordon, David E., et al. 2020. “A SARS-CoV-2Human Protein-Protein Interaction Map Reveals Drug Targets and Potential Drug Repurposing.” bioRxiv). There is a consensus site for the protease (Rut et al. 2020. “Substrate Specificity Profiling of SARS-CoV-2 M^{pro} Protease Provides Basis for Anti-COVID-19 Drug Design.” bioRxiv), though there is variability in the sequences found surrounding the different cleavage sites.

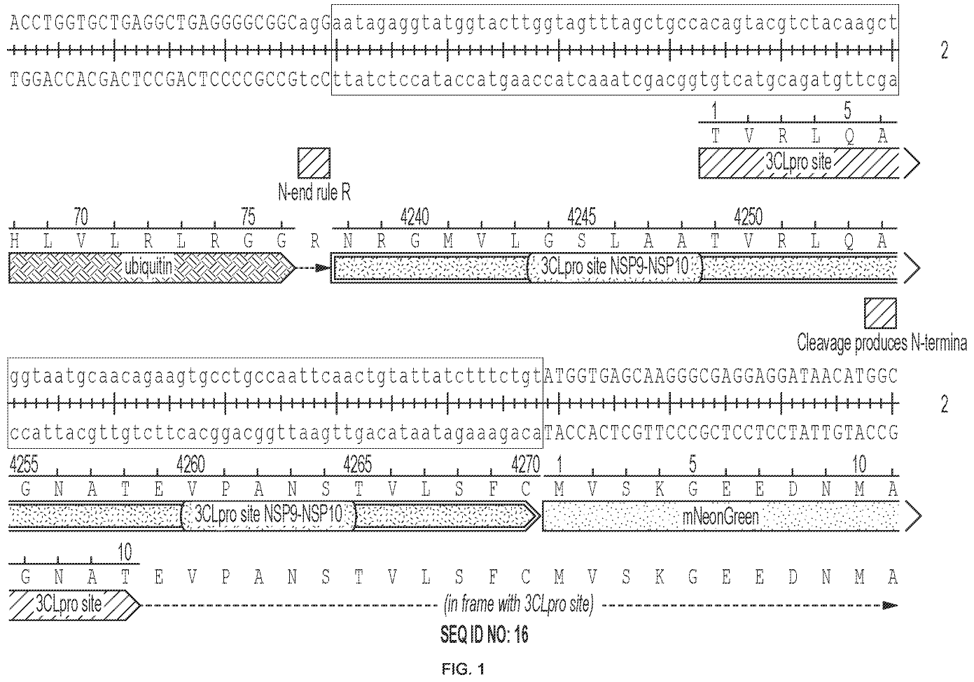

FIG. 1 illustrates an exemplary biosensor specific for 3CL protease of SARS-CoV-2. The sequence features of the biosensor include a ubiquitin domain at the N terminus of the protein, such that cleavage of the ubiquitin domain should leave an arginine at the new N-terminus. The N-terminal arginine functions to shorten the half-life of the protein to a few minutes. Following the ubiquitin domain are the 33 amino acids found between NSP9 and NSP10 in SARS-CoV-2 that serve as the protease cleavage domain. If the 3CL protease cleaves this sequence, the new N-terminus becomes an alanine. The N-terminal alanine greatly enhances the half-life of the remaining reporter protein and mNeonGreen fluorescence can be detected.

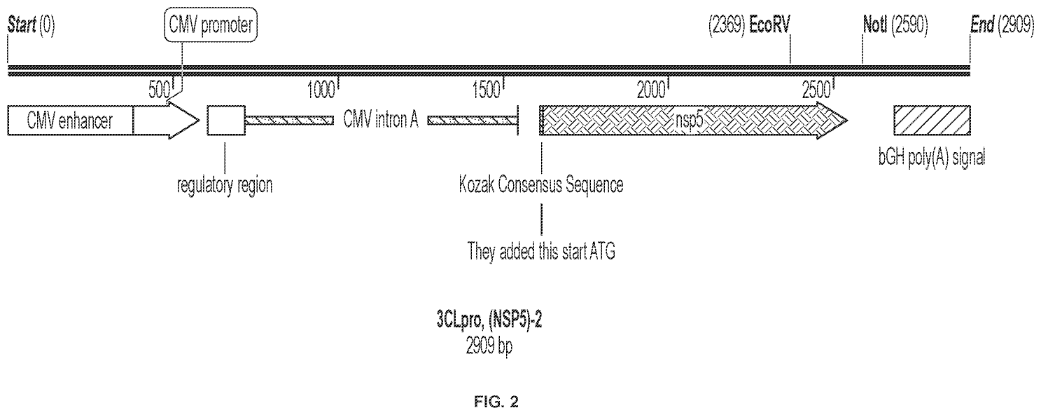

In some embodiments, 3CL protease is co-expressed with a biosensor in a live cell assay to detect compounds that can inhibit 3CL protease, wherein bright fluorescent cells are produced unless a compound can inhibit the 3CL protease. In some embodiments, a construct as shown in FIG. 2 is used to co-express 3CL protease.

Placing different amino acid residues immediately N-terminal to the protease site of the biosensor will create biosensors that degrade at different rates.

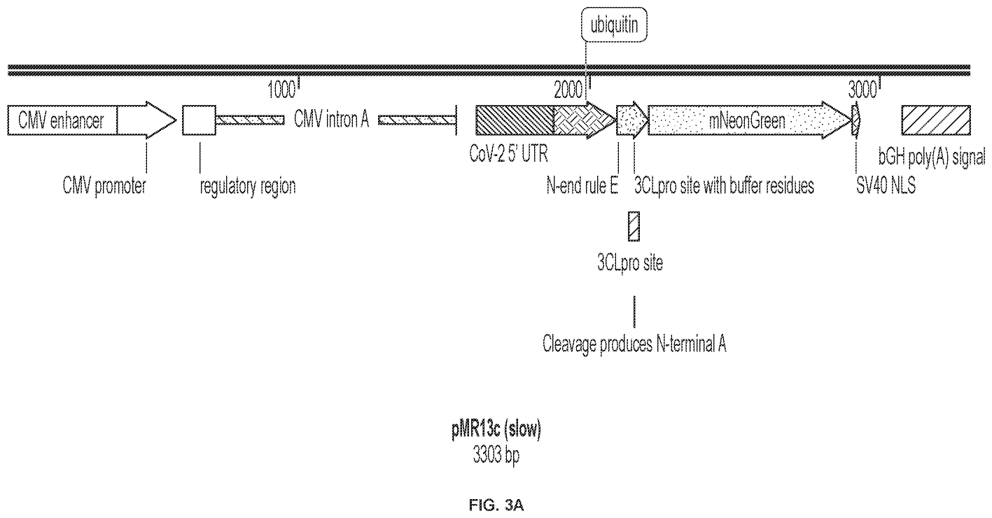

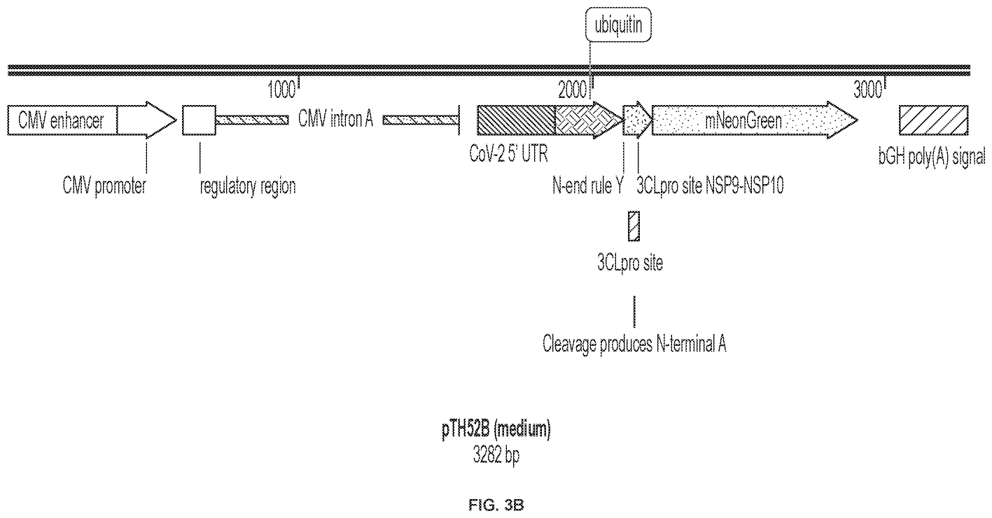

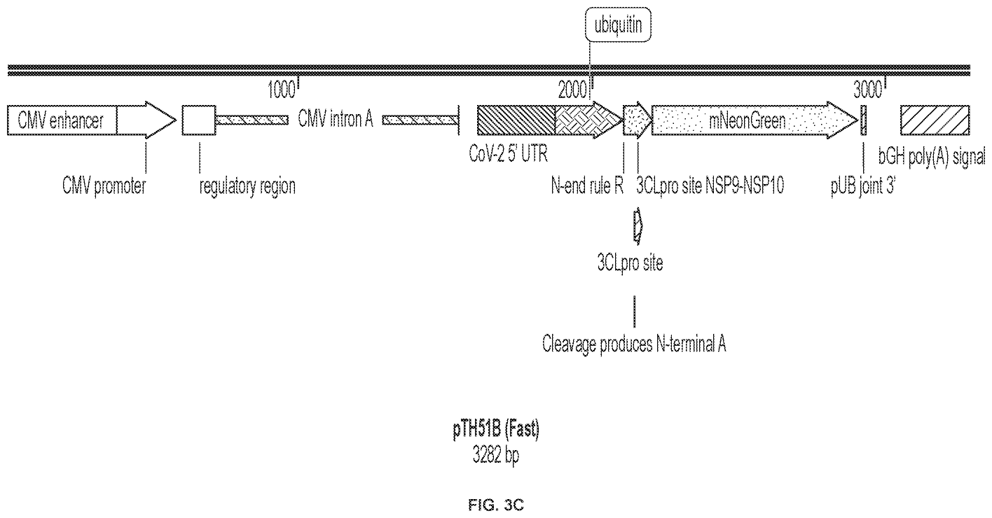

In some embodiments, a 3CL protease biosensor comprises an N-terminal arginine, and degrades quickly. The arginine residue is encoded by nucleotides 2107-2109 of SEQ ID NO: 9. This biosensor is designated the “fast” version and is as shown in FIG. 3C. In some embodiments, a 3CL protease biosensor comprises an N-terminal arginine, which degrades quickly and is encoded by a nucleic acid comprising a nucleotide sequence that is at least 75%, 80%, 85%, 90%, 95%, 96%, 97%, 98%, 99%, or 100% identical to the nucleotide sequence of SEQ ID NO: 9. In some embodiments, a 3CL protease biosensor comprises an N-terminal arginine, which degrades quickly and is encoded by a nucleic acid comprising the nucleotide sequence of SEQ ID NO: 9. In some embodiments, a 3CL protease biosensor comprises an N-terminal arginine, which degrades quickly and is encoded by a nucleic acid consisting of the nucleotide sequence of SEQ ID NO: 9.

In some embodiments, a 3CL protease biosensor contains an N-terminal tyrosine, which degrades at an intermediate rate. The tyrosine residue is encoded by nucleotides 2107-2109 of SEQ ID NO: 8. This biosensor is designated the “medium” version and is shown in FIG. 3B. In some embodiments, a 3CL protease biosensor comprises an N-terminal tyrosine, which degrades at an intermediate rate and is encoded by a nucleic acid comprising a nucleotide sequence that is at least 75%, 80%, 85%, 90%, 95%, 96%, 97%, 98%, 99%, or 100% identical to the nucleotide sequence of SEQ ID NO: 8. In some embodiments, a 3CL protease biosensor comprises an N-terminal tyrosine, which degrades at an intermediate rate and is encoded by a nucleic acid comprising the nucleotide sequence of SEQ ID NO: 8. In some embodiments, a 3CL protease biosensor comprises an N-terminal tyrosine, which degrades at an intermediate rate and is encoded by a nucleic acid consisting of the nucleotide sequence of SEQ ID NO: 8.

In some embodiments, a 3CL protease biosensor contains an N-terminal glutamate, which degrades at an intermediate rate. This biosensor is designated the “slow” version and is shown in FIG. 3A. In some embodiments, the 3CL protease biosensor comprises an N-terminal glutamate, which degrades at a slow rate and is encoded by a nucleic acid comprising the nucleotide sequence of SEQ ID NO: 7. In some embodiments, the 3CL protease biosensor comprises an N-terminal glutamate, which degrades at a slow rate and is encoded by a nucleic acid comprising a nucleotide sequence that is at least 75%, 80%, 85%, 90%, 95%, 96%, 97%, 98%, 99%, or 100% identical to the nucleotide sequence of SEQ ID NO: 7. The glutamate residue is encoded by nucleotides 2107-2109 of SEQ ID NO: 7. In some embodiments, the 3CL protease biosensor comprises an N-terminal glutamate, which degrades at a slow rate and is encoded by a nucleic acid comprising the nucleotide sequence of SEQ ID NO: 7. In some embodiments, the 3CL protease biosensor comprises an N-terminal glutamate, which degrades at a slow rate and is encoded by a nucleic acid consisting of the nucleotide sequence of SEQ ID NO: 7.

SEQ ID NO: 1 is a DNA sequence encoding a 3CL Protease.
SEQ ID NO: 1
TCTGGTTTTAGGAAAATGGCGTTCCCCAGCGGTAAAGTTGAAGGATGTATGGTCCAAGTAACCTGTGGTACCACT
ACCCTTAATGGGCTTTGGTTGGACGACGTAGTCTACTGCCCCCGACACGTAATCTGCACTAGTGAGGATATGCTT
AATCCCAATTACGAAGACCTTTTGATTCGGAAATCCAATCACAACTTCCTGGTCCAAGCGGGCAACGTCCAACTC
AGGGTTATTGGACATAGTATGCAGAATTGCGTACTGAAGCTCAAAGTCGATACTGCAAACCCCAAGACGCCCAAG
TATAAATTCGTCCGAATCCAACCAGGCCAAACATTTTCCGTATTGGCTTGCTATAATGGAAGCCCCAGCGGTGTC
TACCAATGTGCAATGAGACCAAACTTTACGATAAAGGGTTCATTTCTGAACGGCTCTTGCGGTTCCGTTGGTTTT
AACATCGACTATGACTGTGTATCCTTTTGCTACATGCACCATATGGAACTCCCTACCGGTGTCCACGCCGGTACA
GATCTGGAAGGAAATTTCTACGGTCCGTTCGTTGACCGGCAAACCGCGCAAGCGGCTGGAACCGACACAACGATT
ACAGTGAATGTGCTCGCGTGGCTGTACGCAGCAGTCATAAACGGAGACAGGTGGTTTCTGAACCGATTTACGACG
ACTCTCAATGACTTCAACCTTGTTGCGATGAAGTACAATTACGAGCCACTCACCCAGGACCATGTTGATATCCTG
GGTCCCCTCAGTGCCCAGACAGGGATCGCAGTTCTCGATATGTGCGCGTCACTGAAGGAGCTTCTCCAAAATGGA
ATGAATGGGCGGACCATACTTGGTTCCGCACTCCTCGAAGATGAATTTACTCCATTTGACGTGGTCAGACAATGC
AGTGGGGTCACTTTCCAG
SEQ ID NO: 10 is an amino acid sequence for 3CL Protease of SARS-Cov-2.
SEQ ID NO: 10
SGFRKMAFPSGKVEGCMVQVTCGTTTLNGLWLDDVVYCPRHVICTSEDMLNPNYEDLLIRKSNHNFLVQAGNVQLRV
IGHSMONCVLKLKVDTANPKTPKYKFVRIQPGQTFSVLACYNGSPSGVYQCAMRPNFTIKGSFLNGSCGSVGFNIDY
DCVSFCYMHHMELPTGVHAGTDLEGNFYGPFVDRQTAQAAGTDTTITVNVLAWLYAAVINGDRWFLNRFTTTLNDFN
LVAMKYNYEPLTQDHVDILGPLSAQTGIAVLDMCASLKELLQNGMNGRTILGSALLEDEFTPFDVVRQCSGVTFQ
SEQ ID NO: 2 is a DNA sequence for the construct shown in FIG. 2 that
expresses 3CL protease.
SEQ ID NO: 2
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCGTTTAGTGAACCGTC
AGATCGCCTGGAGACGCCATCCACGCTGTTTTGACCTCCATAGAAGACACCGGGACCGATCCAGCCTCCGCGGCC
GGGAACGGTGCATTGGAACGCGGATTCCCCGTGCCAAGAGTGACGTAAGTACCGCCTATAGACTCTATAGGCACA
CCCCTTTGGCTCTTATGCATGCTATACTGTTTTTGGCTTGGGGCCTATACACCCCCGCTTCCTTATGCTATAGGT
GATGGTATAGCTTAGCCTATAGGTGTGGGTTATTGACCATTATTGACCACTCCCCTATTGGTGACGATACTTTCC
ATTACTAATCCATAACATGGCTCTTTGCCACAACTATCTCTATTGGCTATATGCCAATACTCTGTCCTTCAGAGA
CTGACACGGACTCTGTATTTTTACAGGATGGGGTCCCATTTATTATTTACAAATTCACATATACAACAACGCCGT
CCCCCGTGCCCGCAGTTTTTATTAAACATAGCGTGGGATCTCCACGCGAATCTCGGGTACGTGTTCCGGACATGG
GCTCTTCTCCGGTAGCGGCGGAGCTTCCACATCCGAGCCCTGGTCCCATGCCTCCAGCGGCTCATGGTCGCTCGG
CAGCTCCTTGCTCCTAACAGTGGAGGCCAGACTTAGGCACAGCACAATGCCCACCACCACCAGTGTGCCGCACAA
GGCCGTGGCGGTAGGGTATGTGTCTGAAAATGAGCGTGGAGATTGGGCTCGCACGGCTGACGCAGATGGAAGACT
TAAGGCAGCGGCAGAAGAAGATGCAGGCAGCTGAGTTGTTGTATTCTGATAAGAGTCAGAGGTAACTCCCGTTGC
GGTGCTGTTAACGGTGGAGGGCAGTGTAGTCTGAGCAGTACTCGTTGCTGCCGCGCGCGCCACCAGACATAATAG
CTGACAGACTAACAGACTGTTCCTTTCCATGGGTCTTTTCTGCAGTCACCGTCGTCGACACGTGTGATCAGATAT
ACGACTCACTATAGGGAGACCCAAGCTGGCTAGCGTTTGCCGCCACCATGTCTGGTTTTAGGAAAATGGCGTTCC
CCAGCGGTAAAGTTGAAGGATGTATGGTCCAAGTAACCTGTGGTACCACTACCCTTAATGGGCTTTGGTTGGACG
ACGTAGTCTACTGCCCCCGACACGTAATCTGCACTAGTGAGGATATGCTTAATCCCAATTACGAAGACCTTTTGA
TTCGGAAATCCAATCACAACTTCCTGGTCCAAGCGGGCAACGTCCAACTCAGGGTTATTGGACATAGTATGCAGA
ATTGCGTACTGAAGCTCAAAGTCGATACTGCAAACCCCAAGACGCCCAAGTATAAATTCGTCCGAATCCAACCAG
GCCAAACATTTTCCGTATTGGCTTGCTATAATGGAAGCCCCAGCGGTGTCTACCAATGTGCAATGAGACCAAACT
TTACGATAAAGGGTTCATTTCTGAACGGCTCTTGCGGTTCCGTTGGTTTTAACATCGACTATGACTGTGTATCCT
TTTGCTACATGCACCATATGGAACTCCCTACCGGTGTCCACGCCGGTACAGATCTGGAAGGAAATTTCTACGGTC
CGTTCGTTGACCGGCAAACCGCGCAAGCGGCTGGAACCGACACAACGATTACAGTGAATGTGCTCGCGTGGCTGT
ACGCAGCAGTCATAAACGGAGACAGGTGGTTTCTGAACCGATTTACGACGACTCTCAATGACTTCAACCTTGTTG
CGATGAAGTACAATTACGAGCCACTCACCCAGGACCATGTTGATATCCTGGGTCCCCTCAGTGCCCAGACAGGGA
TCGCAGTTCTCGATATGTGCGCGTCACTGAAGGAGCTTCTCCAAAATGGAATGAATGGGCGGACCATACTTGGTT
CCGCACTCCTCGAAGATGAATTTACTCCATTTGACGTGGTCAGACAATGCAGTGGGGTCACTTTCCAGTAACGCG
CCCTCGACTGTGCCTTCTAGTTGCCAGCCATCTGTATCGCGGCCGCTCTAGACCAGGCGCCTGGATCCAGATCAC
TTCTGGCTAATAAAAGATCAGAGCTCTAGAGATCTGTGTGTTGGTTTTTTGTGGATCTGCTGTGCCTTCTAGTTG
CCAGCCATCTGTTGTTTGCCCCTCCCCCGTGCCTTCCTTGACCCTGGAAGGTGCCACTCCCACTGTCCTTTCCTA
ATAAAATGAGGAAATTGCATCGCATTGTCTGAGTAGGTGTCATTCTATTCTGGGGGGTGGGGTGGGGCAGCACAG
CAAGGGGGAGGATTGGGAAGACAATAGCAGGCATGCTGGGGATGCGGTGGGCTCTATGG
SEQ ID NO: 3 is a DNA sequence encoding ubiquitin.
SEQ ID NO: 3
ATGCAGATCTTCGTGAAGACCCTGACCGGCAAGACCATCACCCTGGAGGTGGAGCCCAGCGACACCATCGAGAAC
GTGAAGGCCAAGATCCAGGACAAGGAGGGCATCCCCCCCGACCAGCAGAGGCTGATCTTCGCCGGCAAGCAGCTG
GAGGACGGCAGGACCCTGAGCGACTACAACATCCAGAAGGAGAGCACCCTGCACCTGGTGCTGAGGCTGAGGGGC
GGC
SEQ ID NO: 4 is an amino acid sequence for ubiquitin.
SEQ ID NO: 4
MQIFVKTLTGKTITLEVEPSDTIENVKAKIQDKEGIPPDQQRLIFAGKQLEDGRTLSDYNIQKESTLHLVLR
LRGG
SEQ ID NO: 5 is a DNA sequence encoding a 3CL protease cleavage site.
SEQ ID NO: 5
ACAGTACGTCTACAAGCTGGTAATGCAACA
SEQ ID NO: 6 is an amino acid sequence of a 3CL protease cleavage site.
SEQ ID NO: 6
TVRLQAGNAT
SEQ ID NO: 7 is a DNA sequence encoding the “slow” biosensor shown in FIG.
3A.
SEQ ID NO: 7
GACATTGATTATTGACTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGGTAAACTG
CCCACTTGGCAGTACATCAAGTGTATCATATGCCAAGTACGCCCCCTATTGACGTCAATGACGGTAAATGGCCCG
CCTGGCATTATGCCCAGTACATGACCTTATGGGACTTTCCTACTTGGCAGTACATCTACGTATTAGTCATCGCTA
TTACCATGGTGATGCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAATGTCGTAACAACT
CCGCCCCATTGACGCAAATGGGCGGTAGGCGTGTACGGTGGGAGGTCTATATAAGCAGAGCTCGTTTAGTGAACC
GTCAGATCGCCTGGAGACGCCATCCACGCTGTTTTGACCTCCATAGAAGACACCGGGACCGATCCAGCCTCCGCG
GCCGGGAACGGTGCATTGGAACGCGGATTCCCCGTGCCAAGAGTGACGTAAGTACCGCCTATAGACTCTATAGGC
ACACCCCTTTGGCTCTTATGCATGCTATACTGTTTTTGGCTTGGGGCCTATACACCCCCGCTTCCTTATGCTATA
GGTGATGGTATAGCTTAGCCTATAGGTGTGGGTTATTGACCATTATTGACCACTCCCCTATTGGTGACGATACTT
TCCATTACTAATCCATAACATGGCTCTTTGCCACAACTATCTCTATTGGCTATATGCCAATACTCTGTCCTTCAG
AGACTGACACGGACTCTGTATTTTTACAGGATGGGGTCCCATTTATTATTTACAAATTCACATATACAACAACGC
CGTCCCCCGTGCCCGCAGTTTTTATTAAACATAGCGTGGGATCTCCACGCGAATCTCGGGTACGTGTTCCGGACA
TGGGCTCTTCTCCGGTAGCGGCGGAGCTTCCACATCCGAGCCCTGGTCCCATGCCTCCAGCGGCTCATGGTCGCT
CGGCAGCTCCTTGCTCCTAACAGTGGAGGCCAGACTTAGGCACAGCACAATGCCCACCACCACCAGTGTGCCGCA
CAAGGCCGTGGCGGTAGGGTATGTGTCTGAAAATGAGCGTGGAGATTGGGCTCGCACGGCTGACGCAGATGGAAG
ACTTAAGGCAGCGGCAGAAGAAGATGCAGGCAGCTGAGTTGTTGTATTCTGATAAGAGTCAGAGGTAACTCCCGT
TGCGGTGCTGTTAACGGTGGAGGGCAGTGTAGTCTGAGCAGTACTCGTTGCTGCCGCGCGCGCCACCAGACATAA
TAGCTGACAGACTAACAGACTGTTCCTTTCCATGGGTCTTTTCTGCAGTCACCGTCGTCGACACGTGTGATCAGA
TATACGACTCACTATAGGGAGACCCAAGCTGGCTAGCGTTTATTAAAGGTTTATACCTTCCCAGGTAACAAACCA
ACCAACTTTCGATCTCTTGTAGATCTGTTCTCTAAACGAACTTTAAAATCTGTGTGGCTGTCACTCGGCTGCATG
CTTAGTGCACTCACGCAGTATAATTAATAACTAATTACTGTCGTTGACAGGACACGAGTAACTCGTCTATCTTCT
GCAGGCTGCTTACGGTTTCGTCCGTGTTGCAGCCGATCATCAGCACATCTAGGTTTCGTCCGGGTGTGACCGAAA
GGTAAGATGCAGATCTTCGTGAAGACCCTGACCGGCAAGACCATCACCCTGGAGGTGGAGCCCAGCGACACCATC
GAGAACGTGAAGGCCAAGATCCAGGACAAGGAGGGCATCCCCCCCGACCAGCAGAGGCTGATCTTCGCCGGCAAG
CAGCTGGAGGACGGCAGGACCCTGAGCGACTACAACATCCAGAAGGAGAGCACCCTGCACCTGGTGCTGAGGCTG
AGGGGCGGCGAGAATAGAGGTATGGTACTTGGTAGTTTAGCTGCCACAGTACGTCTACAAGCTGGTAATGCAACA
GAAGTGCCTGCCAATTCAACTGTATTATCTTTCTGTATGGTGAGCAAGGGCGAGGAGGATAACATGGCCTCTCTC
CCAGCGACACATGAGTTACACATCTTTGGCTCCATCAACGGTGTGGACTTTGACATGGTGGGTCAGGGCACCGGC
AATCCAAATGATGGTTATGAGGAGTTAAACCTGAAGTCCACCAAGGGTGACCTCCAGTTCTCCCCCTGGATTCTG
GTCCCTCATATCGGGTATGGCTTCCATCAGTACCTGCCCTACCCTGACGGGATGTCGCCTTTCCAGGCCGCCATG
GTAGATGGCTCCGGATACCAAGTCCATCGCACAATGCAGTTTGAAGATGGTGCCTCCCTTACTGTTAACTACCGC
TACACCTACGAGGGAAGCCACATCAAAGGAGAGGCCCAGGTGAAGGGGACTGGTTTCCCTGCTGACGGTCCTGTG
ATGACCAACTCGCTGACCGCTGCGGACTGGTGCAGGTCGAAGAAGACTTACCCCAACGACAAAACCATCATCAGT
ACCTTTAAGTGGAGTTACACCACTGGAAATGGCAAGCGCTACCGGAGCACTGCGCGGACCACCTACACCTTTGCC
AAGCCAATGGCGGCTAACTATCTGAAGAACCAGCCGATGTACGTGTTCCGTAAGACGGAGCTCAAGCACTCCAAG
ACCGAGCTCAACTTCAAGGAGTGGCAAAAGGCCTTTACCGATGTGATGGGCATGGACGAGCTGTACAAGCCTAAG
AAGAAGAGGAAGGTCTAACGCGCCCTCGACTGTGCCTTCTAGTTGCCAGCCATCTGTATCGCGGCCGCTCTAGAC
CAGGCGCCTGGATCCAGATCACTTCTGGCTAATAAAAGATCAGAGCTCTAGAGATCTGTGTGTTGGTTTTTTGTG
GATCTGCTGTGCCTTCTAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGGTGTCATTCTATTCTGG
GGGGTGGGGTGGGGCAGCACAGCAAGGGGGAGGATTGGGAAGACAATAGCAGGCATGCTGGGGATGCGGTGGGCT
CTATGG
SEQ ID NO: 8 is a DNA sequence encoding the “medium” biosensor shown in FIG.
3B.
SEQ ID NO: 8
GACATTGATTATTGACTAGTTATTAATAGTAATCAATTACGGGGTCATTAGTTCATAGCCCATATATGGAGTTC
CGCGTTACATAACTTACGGTAAATGGCCCGCCTGGCTGACCGCCCAACGACCCCCGCCCATTGACGTCAATAAT
GACGTATGTTCCCATAGTAACGCCAATAGGGACTTTCCATTGACGTCAATGGGTGGAGTATTTACGGTAAACTG
CCCACTTGGCAGTACATCAAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACATCTACGTATTAGTCATCGC
TATTACCATGGTGATGCGGTTTTGGCAGTACATCAATGGGCGTGGATAGCGGTTTGACTCACGGGGATTTCCAA
GTCTCCACCCCATTGACGTCAATGGGAGTTTGTTTTGGCACCAAAATCAACGGGACTTTCCAAAATGTCGTAAC
AACTCCGCCCCATTGACGCAAATGGGCGGTAGGCGTGTACGGTGGGAGGTCTATATAAGCAGAGCTCGTTTAGT
GAACCGTCAGATCGCCTGGAGACGCCATCCACGCTGTTTTGACCTCCATAGAAGACACCGGGACCGATCCAGCC
TCCGCGGCCGGGAACGGTGCATTGGAACGCGGATTCCCCGTGCCAAGAGTGACGTAAGTACCGCCTATAGACTC
TATAGGCACACCCCTTTGGCTCTTATGCATGCTATACTGTTTTTGGCTTGGGGCCTATACACCCCCGCTTCCTT
ATGCTATAGGTGATGGTATAGCTTAGCCTATAGGTGTGGGTTATTGACCATTATTGACCACTCCCCTATTGGTG
ACGATACTTTCCATTACTAATCCATAACATGGCTCTTTGCCACAACTATCTCTATTGGCTATATGCCAATACTC
TGTCCTTCAGAGACTGACACGGACTCTGTATTTTTACAGGATGGGGTCCCATTTATTATTTACAAATTCACATA
TACAACAACGCCGTCCCCCGTGCCCGCAGTTTTTATTAAACATAGCGTGGGATCTCCACGCGAATCTCGGGTAC
GTGTTCCGGACATGGGCTCTTCTCCGGTAGCGGCGGAGCTTCCACATCCGAGCCCTGGTCCCATGCCTCCAGCG
GCTCATGGTCGCTCGGCAGCTCCTTGCTCCTAACAGTGGAGGCCAGACTTAGGCACAGCACAATGCCCACCACC
ACCAGTGTGCCGCACAAGGCCGTGGCGGTAGGGTATGTGTCTGAAAATGAGCGTGGAGATTGGGCTCGCACGGC
TGACGCAGATGGAAGACTTAAGGCAGCGGCAGAAGAAGATGCAGGCAGCTGAGTTGTTGTATTCTGATAAGAGT
CAGAGGTAACTCCCGTTGCGGTGCTGTTAACGGTGGAGGGCAGTGTAGTCTGAGCAGTACTCGTTGCTGCCGCG
CGCGCCACCAGACATAATAGCTGACAGACTAACAGACTGTTCCTTTCCATGGGTCTTTTCTGCAGTCACCGTCG
TCGACACGTGTGATCAGATATACGACTCACTATAGGGAGACCCAAGCTGGCTAGCGTTTATTAAAGGTTTATAC
CTTCCCAGGTAACAAACCAACCAACTTTCGATCTCTTGTAGATCTGTTCTCTAAACGAACTTTAAAATCTGTGT
GGCTGTCACTCGGCTGCATGCTTAGTGCACTCACGCAGTATAATTAATAACTAATTACTGTCGTTGACAGGACA
CGAGTAACTCGTCTATCTTCTGCAGGCTGCTTACGGTTTCGTCCGTGTTGCAGCCGATCATCAGCACATCTAGG
TTTCGTCCGGGTGTGACCGAAAGGTAAGATGCAGATCTTCGTGAAGACCCTGACCGGCAAGACCATCACCCTGG
AGGTGGAGCCCAGCGACACCATCGAGAACGTGAAGGCCAAGATCCAGGACAAGGAGGGCATCCCCCCCGACCAG
CAGAGGCTGATCTTCGCCGGCAAGCAGCTGGAGGACGGCAGGACCCTGAGCGACTACAACATCCAGAAGGAGAG
CACCCTGCACCTGGTGCTGAGGCTGAGGGGCGGCTATAATAGAGGTATGGTACTTGGTAGTTTAGCTGCCACAG
TACGTCTACAAGCTGGTAATGCAACAGAAGTGCCTGCCAATTCAACTGTATTATCTTTCTGTATGGTGAGCAAG
GGCGAGGAGGATAACATGGCCTCTCTCCCAGCGACACATGAGTTACACATCTTTGGCTCCATCAACGGTGTGGA
CTTTGACATGGTGGGTCAGGGCACCGGCAATCCAAATGATGGTTATGAGGAGTTAAACCTGAAGTCCACCAAGG
GTGACCTCCAGTTCTCCCCCTGGATTCTGGTCCCTCATATCGGGTATGGCTTCCATCAGTACCTGCCCTACCCT
GACGGGATGTCGCCTTTCCAGGCCGCCATGGTAGATGGCTCCGGATACCAAGTCCATCGCACAATGCAGTTTGA
AGATGGTGCCTCCCTTACTGTTAACTACCGCTACACCTACGAGGGAAGCCACATCAAAGGAGAGGCCCAGGTGA
AGGGGACTGGTTTCCCTGCTGACGGTCCTGTGATGACCAACTCGCTGACCGCTGCGGACTGGTGCAGGTCGAAG
AAGACTTACCCCAACGACAAAACCATCATCAGTACCTTTAAGTGGAGTTACACCACTGGAAATGGCAAGCGCTA
CCGGAGCACTGCGCGGACCACCTACACCTTTGCCAAGCCAATGGCGGCTAACTATCTGAAGAACCAGCCGATGT
ACGTGTTCCGTAAGACGGAGCTCAAGCACTCCAAGACCGAGCTCAACTTCAAGGAGTGGCAAAAGGCCTTTACC
GATGTGATGGGCATGGACGAGCTGTACAAGAATCGCGCCCTCGACTGTGCCTTCTAGTTGCCAGCCATCTGTAT
CGCGGCCGCTCTAGACCAGGCGCCTGGATCCAGATCACTTCTGGCTAATAAAAGATCAGAGCTCTAGAGATCTG
TGTGTTGGTTTTTTGTGGATCTGCTGTGCCTTCTAGTTGCCAGCCATCTGTTGTTTGCCCCTCCCCCGTGCCTT
CCTTGACCCTGGAAGGTGCCACTCCCACTGTCCTTTCCTAATAAAATGAGGAAATTGCATCGCATTGTCTGAGT
AGGTGTCATTCTATTCTGGGGGGTGGGGTGGGGCAGCACAGCAAGGGGGAGGATTGGGAAGACAATAGCAGGCA
TGCTGGGGATGCGGTGGGCTCTATGG
SEQ ID NO: 9 is a DNA sequence encoding the “fast” biosensor shown in FIG.
3C.
SEQ ID NO: 9
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCGTTTAGTGAACCGTC
AGATCGCCTGGAGACGCCATCCACGCTGTTTTGACCTCCATAGAAGACACCGGGACCGATCCAGCCTCCGCGGCC
GGGAACGGTGCATTGGAACGCGGATTCCCCGTGCCAAGAGTGACGTAAGTACCGCCTATAGACTCTATAGGCACA
CCCCTTTGGCTCTTATGCATGCTATACTGTTTTTGGCTTGGGGCCTATACACCCCCGCTTCCTTATGCTATAGGT
GATGGTATAGCTTAGCCTATAGGTGTGGGTTATTGACCATTATTGACCACTCCCCTATTGGTGACGATACTTTCC
ATTACTAATCCATAACATGGCTCTTTGCCACAACTATCTCTATTGGCTATATGCCAATACTCTGTCCTTCAGAGA
CTGACACGGACTCTGTATTTTTACAGGATGGGGTCCCATTTATTATTTACAAATTCACATATACAACAACGCCGT
CCCCCGTGCCCGCAGTTTTTATTAAACATAGCGTGGGATCTCCACGCGAATCTCGGGTACGTGTTCCGGACATGG
GCTCTTCTCCGGTAGCGGCGGAGCTTCCACATCCGAGCCCTGGTCCCATGCCTCCAGCGGCTCATGGTCGCTCGG
CAGCTCCTTGCTCCTAACAGTGGAGGCCAGACTTAGGCACAGCACAATGCCCACCACCACCAGTGTGCCGCACAA
GGCCGTGGCGGTAGGGTATGTGTCTGAAAATGAGCGTGGAGATTGGGCTCGCACGGCTGACGCAGATGGAAGACT
TAAGGCAGCGGCAGAAGAAGATGCAGGCAGCTGAGTTGTTGTATTCTGATAAGAGTCAGAGGTAACTCCCGTTGC
GGTGCTGTTAACGGTGGAGGGCAGTGTAGTCTGAGCAGTACTCGTTGCTGCCGCGCGCGCCACCAGACATAATAG
CTGACAGACTAACAGACTGTTCCTTTCCATGGGTCTTTTCTGCAGTCACCGTCGTCGACACGTGTGATCAGATAT
ACGACTCACTATAGGGAGACCCAAGCTGGCTAGCGTTTATTAAAGGTTTATACCTTCCCAGGTAACAAACCAACC
AACTTTCGATCTCTTGTAGATCTGTTCTCTAAACGAACTTTAAAATCTGTGTGGCTGTCACTCGGCTGCATGCTT
AGTGCACTCACGCAGTATAATTAATAACTAATTACTGTCGTTGACAGGACACGAGTAACTCGTCTATCTTCTGCA
GGCTGCTTACGGTTTCGTCCGTGTTGCAGCCGATCATCAGCACATCTAGGTTTCGTCCGGGTGTGACCGAAAGGT
AAGATGCAGATCTTCGTGAAGACCCTGACCGGCAAGACCATCACCCTGGAGGTGGAGCCCAGCGACACCATCGAG
AACGTGAAGGCCAAGATCCAGGACAAGGAGGGCATCCCCCCCGACCAGCAGAGGCTGATCTTCGCCGGCAAGCAG
CTGGAGGACGGCAGGACCCTGAGCGACTACAACATCCAGAAGGAGAGCACCCTGCACCTGGTGCTGAGGCTGAGG
GGCGGCAGGAATAGAGGTATGGTACTTGGTAGTTTAGCTGCCACAGTACGTCTACAAGCTGGTAATGCAACAGAA
GTGCCTGCCAATTCAACTGTATTATCTTTCTGTATGGTGAGCAAGGGCGAGGAGGATAACATGGCCTCTCTCCCA
GCGACACATGAGTTACACATCTTTGGCTCCATCAACGGTGTGGACTTTGACATGGTGGGTCAGGGCACCGGCAAT
CCAAATGATGGTTATGAGGAGTTAAACCTGAAGTCCACCAAGGGTGACCTCCAGTTCTCCCCCTGGATTCTGGTC
CCTCATATCGGGTATGGCTTCCATCAGTACCTGCCCTACCCTGACGGGATGTCGCCTTTCCAGGCCGCCATGGTA
GATGGCTCCGGATACCAAGTCCATCGCACAATGCAGTTTGAAGATGGTGCCTCCCTTACTGTTAACTACCGCTAC
ACCTACGAGGGAAGCCACATCAAAGGAGAGGCCCAGGTGAAGGGGACTGGTTTCCCTGCTGACGGTCCTGTGATG
ACCAACTCGCTGACCGCTGCGGACTGGTGCAGGTCGAAGAAGACTTACCCCAACGACAAAACCATCATCAGTACC
TTTAAGTGGAGTTACACCACTGGAAATGGCAAGCGCTACCGGAGCACTGCGCGGACCACCTACACCTTTGCCAAG
CCAATGGCGGCTAACTATCTGAAGAACCAGCCGATGTACGTGTTCCGTAAGACGGAGCTCAAGCACTCCAAGACC
GAGCTCAACTTCAAGGAGTGGCAAAAGGCCTTTACCGATGTGATGGGCATGGACGAGCTGTACAAGAATCGCGCC
CTCGACTGTGCCTTCTAGTTGCCAGCCATCTGTATCGCGGCCGCTCTAGACCAGGCGCCTGGATCCAGATCACTT
CTGGCTAATAAAAGATCAGAGCTCTAGAGATCTGTGTGTTGGTTTTTTGTGGATCTGCTGTGCCTTCTAGTTGCC
AGCCATCTGTTGTTTGCCCCTCCCCCGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTGGGGGGTGGGGTGGGGCAGCACAGCA
AGGGGGAGGATTGGGAAGACAATAGCAGGCATGCTGGGGATGCGGTGGGCTCTATGG
SEQ ID NO: 11 is a DNA sequence encoding the biosensor shown in FIG. 9.
SEQ ID NO: 11
ACATTGATTATTGACTAGTTATTAATAGTAATCAATTACGGGGTCATTAGTTCATAGCCCATATATGGAGTTCCG
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTGATGCGGTTTTGGCAGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCAAAATGTCGTAACAACTCCGC
CCCATTGACGCAAATGGGCGGTAGGCGTGTACGGTGGGAGGTCTATATAAGCAGAGCTCGTTTAGTGAACCGTCA
GATCGCCTGGAGACGCCATCCACGCTGTTTTGACCTCCATAGAAGACACCGGGACCGATCCAGCCTCCGCGGCCG
GGAACGGTGCATTGGAACGCGGATTCCCCGTGCCAAGAGTGACGTAAGTACCGCCTATAGACTCTATAGGCACAC
CCCTTTGGCTCTTATGCATGCTATACTGTTTTTGGCTTGGGGCCTATACACCCCCGCTTCCTTATGCTATAGGTG
ATGGTATAGCTTAGCCTATAGGTGTGGGTTATTGACCATTATTGACCACTCCCCTATTGGTGACGATACTTTCCA
TTACTAATCCATAACATGGCTCTTTGCCACAACTATCTCTATTGGCTATATGCCAATACTCTGTCCTTCAGAGAC
TGACACGGACTCTGTATTTTTACAGGATGGGGTCCCATTTATTATTTACAAATTCACATATACAACAACGCCGTC
CCCCGTGCCCGCAGTTTTTATTAAACATAGCGTGGGATCTCCACGCGAATCTCGGGTACGTGTTCCGGACATGGG
CTCTTCTCCGGTAGCGGCGGAGCTTCCACATCCGAGCCCTGGTCCCATGCCTCCAGCGGCTCATGGTCGCTCGGC
AGCTCCTTGCTCCTAACAGTGGAGGCCAGACTTAGGCACAGCACAATGCCCACCACCACCAGTGTGCCGCACAAG
GCCGTGGCGGTAGGGTATGTGTCTGAAAATGAGCGTGGAGATTGGGCTCGCACGGCTGACGCAGATGGAAGACTT
AAGGCAGCGGCAGAAGAAGATGCAGGCAGCTGAGTTGTTGTATTCTGATAAGAGTCAGAGGTAACTCCCGTTGCG
GTGCTGTTAACGGTGGAGGGCAGTGTAGTCTGAGCAGTACTCGTTGCTGCCGCGCGCGCCACCAGACATAATAGC
TGACAGACTAACAGACTGTTCCTTTCCATGGGTCTTTTCTGCAGTCACCGTCGTCGACACGTGTGATCAGATATA
CGACTCACTATAGGGAGACCCAAGCTGGCTAGCGTTTATTAAAGGTTTATACCTTCCCAGGTAACAAACCAACCA
ACTTTCGATCTCTTGTAGATCTGTTCTCTAAACGAACTTTAAAATCTGTGTGGCTGTCACTCGGCTGCATGCTTA
GTGCACTCACGCAGTATAATTAATAACTAATTACTGTCGTTGACAGGACACGAGTAACTCGTCTATCTTCTGCAG
GCTGCTTACGGTTTCGTCCGTGTTGCAGCCGATCATCAGCACATCTAGGTTTCGTCCGGGTGTGACCGAAAGGTA
AGATGCAGATCTTCGTGAAGACCCTGACCGGCAAGACCATCACCCTGGAGGTGGAGCCCAGCGACACCATCGAGA
ACGTGAAGGCCAAGATCCAGGACAAGGAGGGCATCCCCCCCGACCAGCAGAGGCTGATCTTCGCCGGCAAGCAGC
TGGAGGACGGCAGGACCCTGAGCGACTACAACATCCAGAAGGAGAGCACCCTGCACCTGGTGCTGAGGCTGAGGG
GCGGCAGGAATAGAGGTATGGTACTTGGTAGTTTAGCTGCCACAGTACGTCTACAAGCTGGTAATGCAACAGAAG
TGCCTGCCAATTCAACTGTATTATCTTTCTGTATGGTGAGCAAGGGCGAGGAGGATAACATGGCCTCTCTCCCAG
CGACACATGAGTTACACATCTTTGGCTCCATCAACGGTGTGGACTTTGACATGGTGGGTCAGGGCACCGGCAATC
CAAATGATGGTTATGAGGAGTTAAACCTGAAGTCCACCAAGGGTGACCTCCAGTTCTCCCCCTGGATTCTGGTCC
CTCATATCGGGTATGGCTTCCATCAGTACCTGCCCTACCCTGACGGGATGTCGCCTTTCCAGGACGCCATGGTAG
ATGGCTCCGGATACCAAGTCCATCGCACAATGCAGTTTGAAGATGGTGCCTCCCTTACTGTTAACTACCGCTACA
CCTACGAGGGAAGCCACATCAAAGGAGAGGCCCAGGTGAAGGGGACTGGTTTCCCTGCTGACGGTCCTGTGATGA
CCAACTCGCTGACCGCTGCGGACTGGTGCAGGTCGAAGAAGACTTACCCCAACGACAAAACCATCATCAGTACCT
TTAAGTGGAGTTACACCACTGGAAATGGCAAGCGCTACCGGAGCACTGCGCGGACCACCTACACCTTTGCCAAGC
CAATGGCGGCTAACTATCTGAAGAACCAGCCGATGTACGTGTTCCGTAAGACGGAGCTCAAGCACTCCAAGACCG
AGCTCAACTTCAAGGAGTGGCAAAAGGCCTTTACCGATGTGATGGGCATGGACGAGCTGTACAAGGAGGGCAGAG
GAAGTCTTCTAACATGCGGTGACGTGGAGGAGAATCCCGGGCCTATGGTGTCGAAGGGAGAAGAACTGATTAAGG
AAAACATGCGGATGAAAGTGGTGATGGAAGGGTCGGTGAATGGACACCAATTCAAGTGCACCGGAGAGGGAGAGG
GCAACCCATACATGGGTACTCAGACCATGCGCATCAAGGTCGTTGAAGGAGGACCTCTGCCTTTTGCGTTCGACA
TCCTTGCTACCTCGTTCATGTACGGGTCCCGCACCTTTATCAAGTACCCGAAGGGAATCCCGGATTTCTTCAAGC
AGAGCTTCCCGGAAGGATTCACCTGGGAGAGGGTGACTCGGTACGAAGATGGAGGAGTGCTGACTGCAACCCAAG
ACACTTCGCTCGAGGACGGCTGTCTGGTGTACCATGTCCAAGTGCGGGGTGTGAACTTCCCCTCAAATGGGCCAG
TGATGCAGAAAAAGACCCTCGGATGGGAAGCGAACACCGAGATGATGTACCCGGCGGACGGTGGCTTGCGAGGAT
ACACTCACATGGCCTTGAAGCTGGACGGCGGAGGTCATCTCTCATGCTCCTTTGTCACTACCTACCGCAGCAAGA
AAACTGTCGGAAACATCAAGATGCCGGGCGTGTACTACGTCGATCACCGGCTCGAGAGAATCAAAGAGGCCGACA
AGGAAACGTATGTCGAGCAGCATGAAGTCGCAGTGGCCAGGTACTGCGACCTTCCCTCCAAACTGGGCCACAAGC
TGAATTCTGGCCTGAGAAGCCGCGCACAGGCTTCGAACTCAGCCGTGGATGGGACGGCCGGCCCAGGGTCCACTG
GAAGCAGATAACGCGCCCTCGACTGTGCCTTCTAGTTGCCAGCCATCTGTATCGCGGCCGCTCTAGACCAGGCGC
CTGGATCCAGATCACTTCTGGCTAATAAAAGATCAGAGCTCTAGAGATCTGTGTGTTGGTTTTTTGTGGATCTGC
TGTGCCTTCTAGTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATTCTGGGGGGTGG
GGTGGGGCAGCACAGCAAGGGGGAGGATTGGGAAGACAATAGCAGGCATGCTGGGGATGCGGTGGGCTCTATGG
SEQ ID NO: 12 is a protein sequence of SARS-COV-2 papain-like protease (PL
pro).
SEQ ID NO: 12
EVRTIKVFTTVDNINLHTQVVDMSMTYGQQFGPTYLDGADVTKIKPHNSHEGKTFYVLPNDDTLRVEAFEYYHTT
DPSFLGRYMSALNHTKKWKYPQVNGLTSIKWADNNSYLATALLTLQQIELKFNPPALQDAYYRARAGEAANFCAL
ILAYCNKTVGELGDVRETMSYLFQHANLDSCKRVLNVVCKTCGQQQTTLKGVEAVMYMGTLSYEQFKKGVQIPCT
CGKQATKYLVQQESPFVMMSAPPAQYELKHGTFTCASEYTGNYQCGHYKHITSKETLYCIDGALLTKSSEYKGPI
TDVFYKENSYTTTIKPVTY
SEQ ID NO: 13 is a protein sequence of an mNeonGreen fluorescent protein
that can be used as a reporter protein in a biosensor of the present
disclosure.
SEQ ID NO: 13
MVSKGEEDNMASLPATHELHIFGSINGVDFDMVGQGTGNPNDGYEELNLKSTKGDLQFSPWILVPHIGYGFHQYL
PYPDGMSPFQDAMVDGSGYQVHRTMQFEDGASLTVNYRYTYEGSHIKGEAQVKGTGFPADGPVMTNSLTAADWCR
SKKTYPNDKTIISTFKWSYTTGNGKRYRSTARTTYTFAKPMAANYLKNQPMYVFRKTELKHSKTELNFKEWQKAF
TDVMGMDELYK
SEQ ID NO: 14 is a protein sequence of a red fluorescent protein that can
be used as a reporter protein in a biosensor of the present disclosure.
SEQ ID NO: 14
MVSKGEELIKENMRMKVVMEGSVNGHQFKCTGEGEGNPYMGTQTMRIKVVEGGPLPFAFDILATSFMYGSRTFIK
YPKGIPDFFKQSFPEGFTWERVTRYEDGGVLTATQDTSLEDGCLVYHVQVRGVNFPSNGPVMQKKTLGWEANTEM
MYPADGGLRGYTHMALKLDGGGHLSCSFVTTYRSKKTVGNIKMPGVYYVDHRLERIKEADKETYVEQHEVAVARY
CDLPSKLGHKLNSGLRSRAQASNSAVDGTAGPGSTGSR
SEQ ID NO: 15 is a protein sequence of a T2A peptide signal that can be
used as a self-cleaving peptide in a biosensor of the present disclosure.
SEQ ID NO: 15
EGRGSLLTCGDVEENPGP
SEQ ID NO: 16 is a partial DNA sequence of a biosensor of the present
disclosure. SEQ ID NO: 16 is shown in FIG. 1.
SEQ ID NO: 16
acctggtgctgaggctgaggggcggcaggaatagaggtatggtactggtagtttagctgccacagtacgtctaca
agctggtaatgcaacagaagtgcctgccaattcaactgtattatctttctgtatggtgagcaagggcgaggagga
taacatggc
SEQ ID NO: 17 is a partial amino acid sequence of a biosensor of the present
disclosure. SEQ ID NO: 17 is shown in FIG. 1.
SEQ ID NO: 17
HLVLRLRGGRNRGMVLGSLAATVRLQAGNATEVPANSTVLSFCMVSKGEEDNMA
SEQ ID NO: 23 is a partial nucleotide sequence of a biosensor of the present
disclosure. SEQ ID NO: 23 encodes an exemplary sequence for a 5′UTR-degron-
cleavage site portion of the biosensor.
SEQ ID NO: 23
attaaaggtttataccttcccaggtaacaaaccaaccaactttcgatctcttgtagatctgttctctaaacgaac
tttaaaatctgtgtggctgtcactcggctgcatgcttagtgcactcacgcagtataattaataactaattactgt
cgttgacaggacacgagtaactcgtctatcttctgcaggctgcttacggtttcgtccgtgttgcagccgatcatc
agcacatctaggtttcgtccgggtgtgaccgaaaggtaagatgcagatcttcgtgaagaccctgaccggcaagac
catcaccctggaggtggagcccagcgacaccatcgagaacgtgaaggccaagatccaggacaaggagggcatccc
ccccgaccagcagaggctgatcttcgccggcaagcagctggaggacggcaggaccctgagcgactacaacatcca
gaaggagagcaccctgcacctggtgctgaggctgaggggcggcnnnnnnnnnnnnnnnnnnnnnnnnnnnnnnnn
nnnnacagtacgtctacaagctggtaatgcaacannnnnnnnnnnnnnnnnnnnnnnnnnnnnnnnnnnn
SEQ ID NO: 24 is a partial nucleotide sequence of a biosensor of the present
disclosure. SEQ ID NO: 24 encodes an exemplary sequence for a degron-cleavage
site portion of the biosensor.
SEQ ID NO: 24
atgcagatcttcgtgaagaccctgaccggcaagaccatcaccctggaggtggagcccagcgacaccatcgagaac
gtgaaggccaagatccaggacaaggagggcatcccccccgaccagcagaggctgatcttcgccggcaagcagctg
gaggacggcaggaccctgagcgactacaacatccagaaggagagcaccctgcacctggtgctgaggctgaggggc
ggcnnnnnnnnnnnnnnnnnnnnnnnnnnnnnnnnnnnnacagtacgtctacaagctggtaatgcaacannnnnn
nnnnnnnnnnnnnnnnnnnnnnnnnnnnnn
SEQ ID NO: 25 is a partial amino acid sequence of a biosensor of the present
disclosure. SEQ ID NO: 25 encodes an exemplary sequence for a degron-cleavage
site portion of the biosensor.
SEQ ID NO: 25
MQIFVKTLTGKTITLEVEPSDTIENVKAKIQDKEGIPPDQQRLIFAGKQLEDGRTLSDYN
IQKESTLHLVLRLRGGXXXXXXXXXXXXTVRLQAGNATXXXXXXXXXXXX

### Detection of Viral Replication

In some embodiments, the biosensors disclosed herein can be used in the detection of replication of an organism. The organism can be any organism that expresses a protease, including but not limited to viruses, bacteria, and mammalian cells. In some embodiments, replication of an organism, such as a virus, is detected in a sample of living cells.

In some embodiments, replication of an organism is detected by a protease produced by that organism cleaving a protease cleavage site positioned 3′ to a degron and 5′ to a reporter protein, thereby extending the half-life of the reporter protein such that the reporter protein is then detected.

In coronaviruses, such as the human coronaviruses HCoV-229E, HCoV-0C43, SARS-CoV, HCoV-NL63, human coronavirus HKU1, MERS-CoV, or SARS-CoV-2, 3CL protease is an essential gene, and its activity is crucial to viral replication. Therefore, any cell supporting coronavirus replication will have 3CL protease expression that can be detected using the biosensor described herein.

In some embodiments, replication of coronaviruses, such as human coronaviruses, (e.g., SARS-CoV-2) is detected by 3CL protease cleaving a 3CL protease cleavage site positioned 3′ to a degron and 5′ to a reporter protein, thereby extending the half-life of the reporter protein such that the reporter protein is then detected in a live cell assay. In some embodiments, the reporter protein detected in a live cell assay is a fluorescent protein. In some embodiments, the reporter protein detected in a live cell assay is mNeonGreen.

In some embodiments, replication of coronaviruses, such as human coronaviruses, (e.g., SARS-CoV-2) is detected by PL protease cleaving a PL protease cleavage site positioned 3′ to a degron and 5′ to a reporter protein, thereby extending the half-life of the reporter protein such that the reporter protein is then detected in a live cell assay. In some embodiments, the reporter protein detected in a live cell assay is a fluorescent protein. In some embodiments, the reporter protein detected in a live cell assay is mNeonGreen.

In some embodiments, apoptosis of mammalian cells is detected by a caspase cleaving a caspase cleavage site positioned 3′ to a degron and 5′ to a reporter protein, thereby extending the half-life of the reporter protein such that the reporter protein is then detected in a live cell assay. In some embodiments, when a caspase is present, mammalian cells are undergoing apoptosis and the presence of the reporter protein will indicate the same. In some embodiments, the reporter protein detected in a live cell assay is a fluorescent protein. In some embodiments, the reporter protein detected in a live cell assay is mNeonGreen.

A person of ordinary skill in the art will appreciate that the biosensors disclosed herein can be applied in a live cell assay of a variety of cellular samples. In some embodiments, the samples are from patients. In some embodiments, the samples are from patients and a live cell assay is used to detect the presence of SARS-CoV-2. In some embodiments, the samples are from cultured cells. In some embodiments, the samples are from cultured cells and a live cell assay is used to detect the presence of SARS-CoV-2. In some embodiments, the samples are from wastewater. In some embodiments, the samples are from wastewater and a live cell assay is used to detect the presence of SARS-CoV-2.

### Assay to Detect Inhibitors

In some embodiments, the biosensors disclosed herein can be used to detect compounds that inhibit the replication of an organism. In some embodiments, a biosensor is used to detect compounds that inhibit viral replication. In some embodiments, viral replication is inhibited by inhibiting a viral protease. In some embodiments, a biosensor is used in a high throughput inhibitor assay.

In some embodiments, the biosensor is used to detect compounds that will inhibit SARS-CoV-2. In some embodiments, 3CL protease is co-expressed with a biosensor in a live cell assay, wherein bright fluorescent cells are produced unless a compound can inhibit the 3CL protease. In some embodiments, a construct as shown in FIG. 2 is used to co-express 3CL protease. In some embodiments, the construct used to express 3CL protease comprises the nucleotide sequence of SEQ ID NO: 2. Since viral replication depends on 3CL protease, anything that successfully blocks SARS-CoV-2 viral entry or viral replication will be detectable with the 3CL protease biosensor.

In some embodiments a second reporter protein could be co-expressed to detect toxic compounds that kill cells in the sample. Such a dual color read out would make it possible to screen a million compounds to identify drugs that block the protease but do not kill mammalian cells. In some embodiments, the second reporter protein produces a red fluorescent signal. In some embodiments, expression of the second reporter protein can be accomplished from the same biosensor as the first reporter protein. In some embodiments, the two protein ORFs can be separated by a self-cleaving peptide sequence. In some embodiments, the biosensor can be encoded by the vector of FIG. 9. In some embodiments, the biosensor can be encoded by the sequence of SEQ ID NO: 11.

In some embodiments, the biosensor is used to detect compounds that will inhibit SARS-CoV-2. In some embodiments, PL protease is co-expressed with a biosensor in a live cell assay, wherein bright fluorescent cells are produced unless a compound can inhibit the PL protease. Since viral replication depends on PL protease, anything that successfully blocks SARS-CoV-2 viral entry or viral replication will be detectable with the PL protease biosensor.

In some embodiments, the biosensor is used to detect compounds that will inhibit mammalian apoptosis. In some embodiments, a caspase is co-expressed with a biosensor in a live cell assay, wherein bright fluorescent cells are produced unless a compound can inhibit the caspase.

### Delivery Systems

Biosensors can be packaged in a delivery system to achieve more consistent expression when delivered to a cellular sample. In some embodiments a viral delivery system is used to deliver 3CL protease biosensors to a cellular sample. In some embodiments a viral delivery system is used to deliver 3CL protease to a cellular sample.

Viral delivery systems that can be used include, but are not limited to, adenovirus vectors, retrovirus vectors, adeno-associated virus vectors, and poxvirus, e.g., vaccinia virus vectors, baculovirus vectors, or herpesvirus vectors. In some embodiments, a non-viral delivery system is used. Other delivery systems include plasmids, liposomes, electrically charged lipids (cytofectins), DNA-protein complexes, and biopolymers.

Baculovirus gene transfer into mammalian cells, known as BacMam, is the use of baculovirus to deliver genes to mammalian cells. BacMam viral delivery makes it possible to optimize an assay by systematically varying the relative expression levels of different components.

In some embodiments, a BacMam viral delivery system is used to deliver 3CL protease biosensors to sample cells. In some embodiments, the amount of delivered BacMam expressing the 3CL protease biosensor is varied to optimize the expression of the 3CL protease biosensor. In some embodiments, between about 1 μl and about 10 μl of BacMam expressing the 3CL protease biosensor is used for delivery. In some embodiments, about 1 about 2 about 3 about 4 about 5 about 6 about 7 about 8 about 9 or about 10 pl of BacMam expressing the 3CL protease biosensor is used for delivery.

In some embodiments, a BacMam viral vector encoding a biosensor is delivered to cells. In some embodiments, about 1×10¹⁰, 2×10¹⁰, or 3×10¹⁰ viral genomes per mL are delivered to the cells. In some embodiments, about 1×10⁸, 2×10⁸, or 3×10⁸ infectious units per mL are delivered to the cells.

In some embodiments, a BacMam viral delivery system is used to deliver 3CL protease to a cellular sample. In some embodiments, the amount of delivered BacMam expressing the 3CL protease is varied to optimize the expression of the 3CL protease. In some embodiments, between about 0.5 pl and 10 pl of BacMam expressing 3CL protease is used for delivery. In some embodiments, about 0.5 about 1 about 1.5 about 2 about 2.5 about 3 μl, about 3.5 μl, about 4 μl, about 4.5 μl, about 5 μl, about 4.5 μl, about 5 μl, about 5.5 μl, about 6 about 6.5 about 7 about 7.5 about 8 about 8.5 about 9 about 9.5 or about 10 μ.1 of BacMam expressing 3CL protease is used for delivery.

In some embodiments, more than one BacMam virus is used, each expressing different proteins. In some embodiments, a mixture of two BacMam viruses, one that expresses 3CL protease and one that expresses the fluorescent 3CL protease biosensor are used. In some embodiments, the amount of delivered BacMam expressing the 3CL protease is varied to optimize the expression of the 3CL protease and the amount of delivered BacMam expressing the 3CL protease biosensor is varied to optimize the expression of the 3CL protease biosensor.

### Protein Expression Systems

The polypeptides of the invention can also be expressed in bacteria or yeast or plant cells. In this regard it will be appreciated that various unicellular non-mammalian microorganisms such as bacteria can also be transformed; i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae.

Alternatively, polynucleotide sequences of the invention can be incorporated in transgenes for introduction into the genome of a transgenic animal (see, e.g., Deboer et al., U.S. Pat. No. 5,741,957, Rosen, U.S. Pat. No. 5,304,489, and Meade et al., U.S. Pat. No. 5,849,992).

In one embodiment, the host cell is a eukaryotic cell. As used herein, a eukaryotic cell refers to any animal or plant cell having a definitive nucleus. Eukaryotic cells of animals include cells of vertebrates, e.g., mammals, and cells of invertebrates, e.g., insects. Eukaryotic cells of plants specifically can include, without limitation, yeast cells. A eukaryotic cell is distinct from a prokaryotic cell, e.g., bacteria.

In certain embodiments, the eukaryotic cell is a mammalian cell. A mammalian cell is any cell derived from a mammal. Mammalian cells specifically include, but are not limited to, mammalian cell lines. In one embodiment, the mammalian cell is a human cell. In another embodiment, the mammalian cell is a HEK 293 cell, which is a human embryonic kidney cell line. HEK 293 cells are available as CRL-1533 from American Type Culture Collection, Manassas, VA, and as 293-H cells, Catalog No. 11631-017 or 293-F cells, Catalog No. 11625-019 from Invitrogen (Carlsbad, Calif). In some embodiments, the mammalian cell is a PER. C6® cell, which is a human cell line derived from retina. PER. C6® cells are available from Crucell (Leiden, The Netherlands). In other embodiments, the mammalian cell is a Chinese hamster ovary (CHO) cell. CHO cells are available from American Type Culture Collection, Manassas, VA. (e.g., CHO-K1; CCL-61). In still other embodiments, the mammalian cell is a baby hamster kidney (BHK) cell. BHK cells are available from American Type Culture Collection, Manassas, Va. (e.g., CRL-1632). In some embodiments, the mammalian cell is a HKB11 cell, which is a hybrid cell line of a HEK293 cell and a human B cell line. Mei et al., Mol. Biotechnol. 34(2): 165-78 (2006).

### EXAMPLES

While several experimental Examples are contemplated, these Examples are intended non-limiting.

### Example 1: Optimizing Degradation Rates of Prototype Biosensors

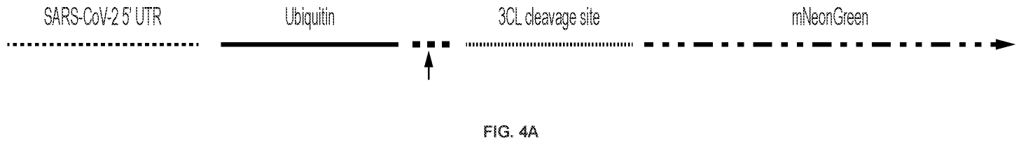

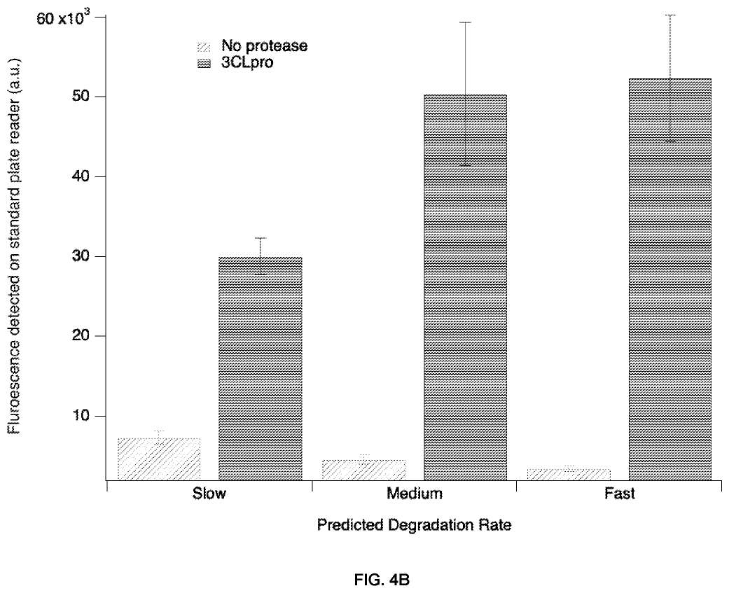

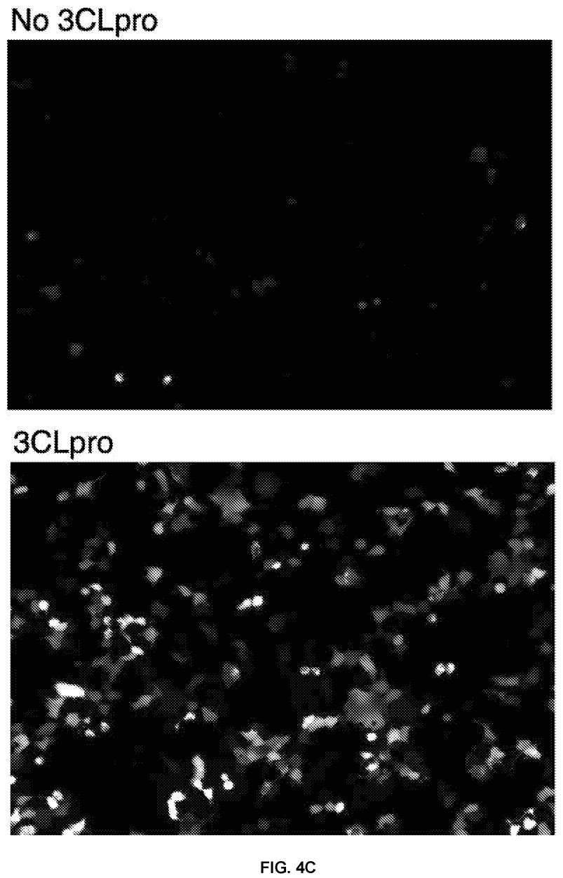

In optimizing a biosensor, various rates need to be taken into account. First, the time it takes to fold the reporter protein mNeonGreen, and the fluorophore formation rate is known. Second, the processivity of the 3CL protease in mammalian cells is unknown. Since the reporter signal depends on both the rate at which the reporter is produced, and the rate in which it is protected from the degron by the 3CL protease, three versions of the 3CL protease biosensor were created. These biosensors included either an R, A, or E amino acid at the N-terminus produced by de-ubiquitination. According to the N-end rule, these N-termini produce proteins that are degraded at different rates. The first test 3CL protease biosensor contained an N-terminal R, which degrades quickly, and was designated the “fast” version, as shown in FIG. 3C. The second test 3CL protease biosensor contained an N-terminal A, which degrades at an intermediate rate, and was designated the “medium” version, as shown in FIG. 3B. The third test 3CL protease biosensor contained N-terminal E, which degrades at a slow rate, and was designated the “slow” version, as shown in FIG. 3A. A schematic of the sequence features of all biosensors tested is presented in FIG. 4A.

In order to test the prototype biosensors with different predicted degradation rates, HEK293 cells were transiently transfected with plasmids encoding the 3CL protease and one of the 3CL protease biosensor prototypes. As a control, adjacent wells on the plate contained the 3CL protease biosensor with no protease. Twenty-four hours after the transfection, the cells were washed in PBS and then the fluorescence of each well was collected on a BioTek Synergy fluorescence plate reader.

As shown in FIG. 4B, all three test 3CL protease biosensor prototypes produced a bright fluorescent response if the 3CL protease was present, and showed very little fluorescence if the 3CL protease was absent. The contrast between the wells with and without 3CL protease was greatest with the “fast” biosensor. Thus, the “fast” biosensor produced the highest level of fluorescence over the baseline fluorescence of the control.

### Example 2: BacMam Viral Delivery of 3CL Protease and 3CL Protease Biosensors

BacMam viral delivery makes it possible to optimize an assay by systematically varying the relative expression levels of different components. The following protocol was used to optimize BacMam viral delivery of the 3CL protease and 3CL protease biosensors. On day one, HEK 293T cells were plated in a 96 well plate at 50,000 cells per well. The following day BacMam viruses were added to the well to express a 3CL protease biosensor and the 3CL protease. To express the biosensor, each well received 5 μl of virus (2×10¹⁰) expressing either the fast or medium rate biosensor. The amount of BacMam expressing the protease was systematically varied from 5 μl to 1.25 μl of virus, or no-virus control. On day three, the cells were washed with PBS and the fluorescence was measured on a BioTek Synergy plate reader.

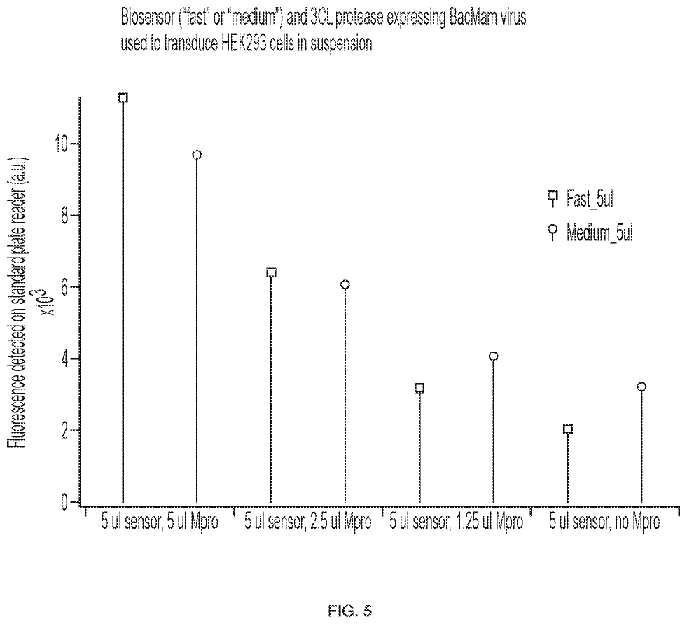

The data obtained is presented in FIG. 5. Both of the tested biosensors, the fast and medium rate biosensors, reported the presence of 3CL protease activity in a dose dependent manner. The fast biosensor showed a steeper fluorescence/3CL protease dependence, indicating that it is the most sensitive of the biosensors to protease activity levels.

### Example 3: Live Cell Assay for Protease Inhibitors

Replication of the SARS-CoV-2 virus depends crucially on the activity of its main protease, 3CL protease (3CLpro). This dependence is in many ways the Achilles heel of the virus: without 3CLpro it cannot replicate and it is harmless. Different versions of the 3CL protease can be found in many coronaviruses, including the feline infectious peritonitis virus (FPIV). When cats demonstrate the clinical manifestations that indicate they have FPIV, they are destined to die; it is 100% lethal. However, an inhibitor to the FPIV version of 3CL protease rescues them (Kim, et al. 2016. “Reversal of the Progression of Fatal Coronavirus Infection in Cats by a Broad-Spectrum Coronavirus Protease Inhibitor.” PLoS Pathogens 12 (3): e1005531). This inhibitor, GC376, is currently marketed by Anivive Lifesciences for use in cats. The exciting news is that two groups have discovered that GC376 can also inhibit the human SARS-CoV-2 3CL protease, which is incredibly promising (Iketani, et al. 2020. “Lead Compounds for the Development of SARS-CoV-2 3CL Protease Inhibitors.” bioRxiv: The Preprint Server for Biology, August; Hung, et al. 2020. “Discovery of M Protease Inhibitors Encoded by SARS-CoV-2.” Antimicrobial Agents and Chemotherapy, July). To verify the activity of GC376, a 3CL protease live cell assay was performed which includes both the SARS-CoV-2 3CL protease, as well as, the 3CL protease biosensor. The results were consistent with the in vitro measurements of Iketani and colleagues (Iketani, et al. 2020. “Lead Compounds for the Development of SARS-CoV-2 3CL Protease Inhibitors.” bioRxiv: The Preprint Server for Biology, August).

The following live cell assay protocol was developed to screen for protease inhibitors using a protease biosensor:
Day 1: 50,000 HEK 293 cells per well are plated in standard media in 96 well plates and grown overnight in DMEM with 10% Fetal Bovine Serum in a humidified, 37° incubator with 5% CO2.
Day 2: Transduction mix is prepared with two BacMam viruses. One virus delivers an optimized amount of 3CL protease, the other delivers the protease biosensor. The mix also includes sodium butyrate, an HDAC inhibitor that promotes expression. Once the transduction mix has been added to the wells, compounds and suspected protease inhibitors are added to the wells. The control wells contain no protease or known inhibitors.
Day 3: The following day, 10 to 24 hours after the transduction step, the cells are washed with phosphate buffered saline (PBS) to remove autofluorescent media, and fluorescence is measured using standard fluorescence plate readers. The samples are then analyzed for fluorescence, and the case of no inhibitors, the cells should be barely fluorescent if at all. In the case where inhibitors are present, the inhibitors should produce fluorescence in a dose-dependent manner.

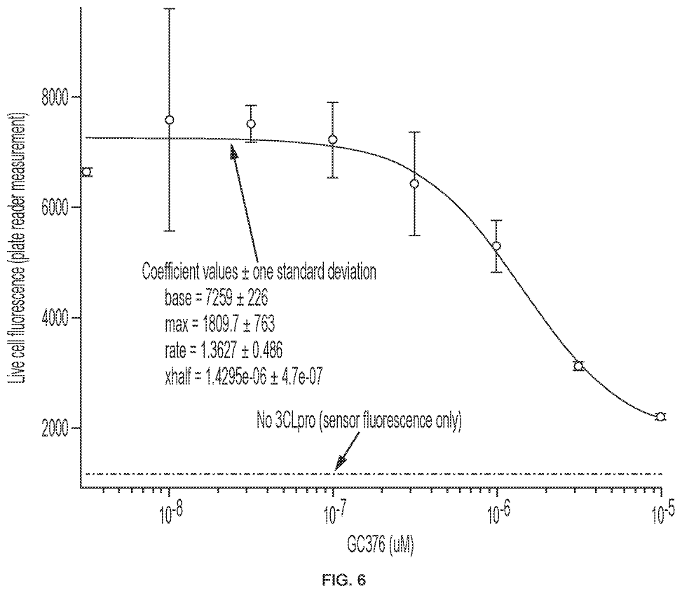

The protocol above was used to test if GC376 (Anivive Lifesciences) inhibition of SARS-CoV-2 3CL protease activity could be detected in living cells. 3CL protease was co-expressed with the fast 3CL protease biosensor. HEK 293 cells were plated in a 96 well plate on day one. On day two, the BacMam viruses expressing 3CL protease and the 3CL protease fast biosensor were added to the wells, as well as a dilution series of GC376. As shown in FIG. 6, dose dependent inhibition of the 3CL protease activity by GC376 was observed.

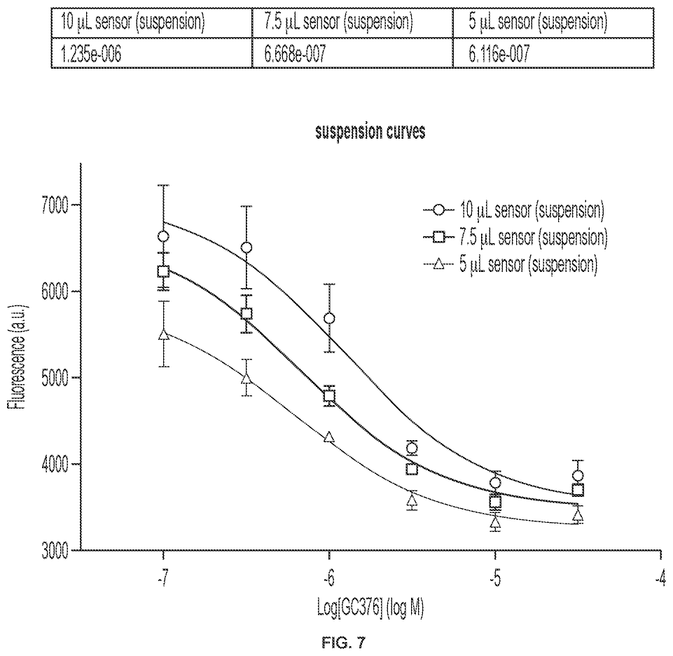

Further testing was done to determine the effect of varying the amount of 3CL protease biosensor in the GC376 dosing assay. As shown in FIG. 7, HEK293 cells were transduced with BacMam viruses expressing 3CL protease and different amounts of the 3CL protease biosensor (5 μl, 7.5 μl, and 10 μl). GC376 was added to the cells to test its efficacy in blocking the 3CL protease. The following day the 96 well plate was washed with PBS to remove autofluorescent media, and then the fluorescence within the living cells was read on a conventional BioTek Synergy fluorescence plate reader. The resulting fluorescence/dose relationships for GC376, shown in FIG. 7, are well fit by Hill functions with EC₅₀ values of 600 nM to 1.2 μM, depending upon the amount of biosensor expression.

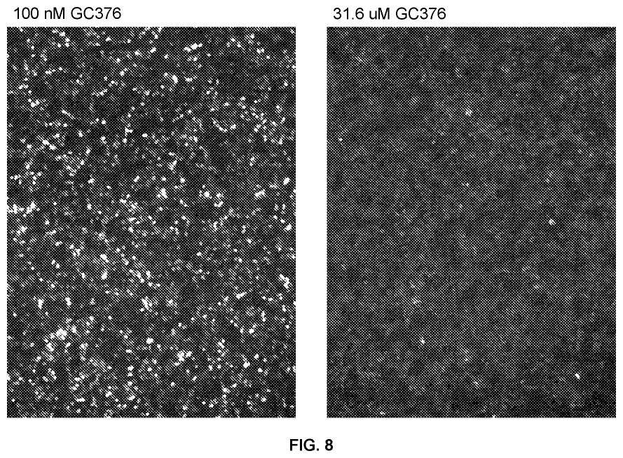

As shown in FIG. 8, images were collected from wells of green fluorescent, living HEK293 cells incubated overnight in different concentrations of GC376, either 100 nM or 31.6 μM. The difference in fluorescence intensity is due to differences in 3CL protease activity. A suspension of HEK 293 cells (480,000 cells/ml) was transduced with a mixture of two BacMam viruses, one that expresses 3CL protease, the other expressed the fluorescent 3CL protease biosensor, as well as the HDAC inhibitor, sodium butyrate, at 2 mM. This transduction mix with cells was plated 100 μl per well, in a 96 well dish, and incubated for 5 hours to let the cells attach to the plate. The cells were then washed 3× with fresh media to remove the BacMam viruses, and fresh media with 2 mM sodium butyrate and different concentrations of the inhibitor GC376 was added to each well. Twenty hours later, the cells were washed with PBS to remove auto-fluorescent media and the imaged on a BioTek Lion Heart imaging plate reader with a 4X objective lens and identical acquisition settings for every well. As shown in FIG. 8, a higher level of fluorescence is observed at 100 nM of GC376 than at 31.6 μM GC376, indicating the ability of the higher concentration of GC376 to inhibit 3CL protease.

This example confirms that the 3CL protease biosensor can be used in a live cell assay to detect inhibitors of 3CL protease.

### Example 4: Live Cell Assay for SARS-CoV-2 Viral Replication

The following protocol was developed to detect SARS-CoV-2 viral replication using a protease biosensor in a live cell assay:

Day 1: Vero 6 cells, or another cell line that can support viral entry and replication, are plated at 30,000 cells per well in standard media in 96 well plates and grown overnight in a humidified, 37° incubator with 5% CO2.

Day 2: The cells are transduced with the BacMam virus that expresses the 3CL protease biosensor. The mix also includes sodium butyrate, an HDAC inhibitor that promotes expression. Once the transduction mix has been added to the wells, sterile filtered (0.2 μM filter) inoculum is added to the wells. Control wells include no 3CL protease and 3CL protease of varying amounts.

Day 3: Twelve hours after the inoculum is added to the culture, the plate is inserted into an environmental chamber to monitor the accumulation of fluorescence over time. The presence of significant fluorescence in the well indicates the presence of a replicating virus, and the rate at which the fluorescence grows exponentially over time is a measurement of the amount of virus that was introduced into the well.

### Example 5: Two-Reporter Vector for Detecting 3CL Protease Activity

FIG. 9 illustrates an alternative embodiment of a 3CLpro biosensor. The 5′ UTR of the SARS-CoV-2 virus is at the 5′ end of the transcript, to ensure that the sensor is expressed even in cells infected with live SARS-CoV-2 virus (Tanaka et al., 2012. J. Virol. 86, 11128-11137; Zhang et al., 2021. Sci. Adv. 7.). A degron is positioned at the beginning of the N-terminus of the translated protein such that de-ubiquitination will leave an arginine at the amino terminus, ensuring that the sensor will be degraded before it can become fluorescent (Houser et al., 2012.). A 3CLpro cleavage site is positioned between the N-terminal degron and the remaining protein such that 3CLpro digestion will rescue the fluorescent protein from degradation and lead to a detectable fluorescent signal. A T2A peptide is included at the C-terminus of the green protein so that a red protein is translated independently of the green one (Szymczak et al., 2004. Correction of multi-gene deficiency in vivo using a single “self-cleaving” 2A peptide-based retroviral vector. Nature Biotechnology 22, 589-594). The red fluorescence from this constitutively expressed protein can be used as an independent indicator of cell health and/or transduction efficiency. The vector delivering the 3CLpro biosensor comprises the nucleotide sequence of SEQ ID NO: 11.

### Example 6: Transduction of Cells Using the Two-Reporter Vector

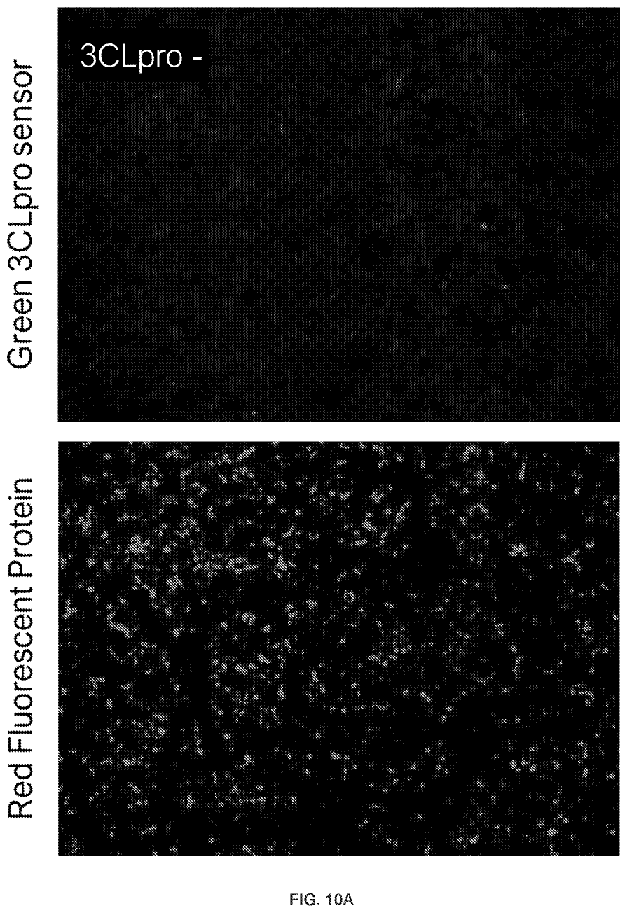

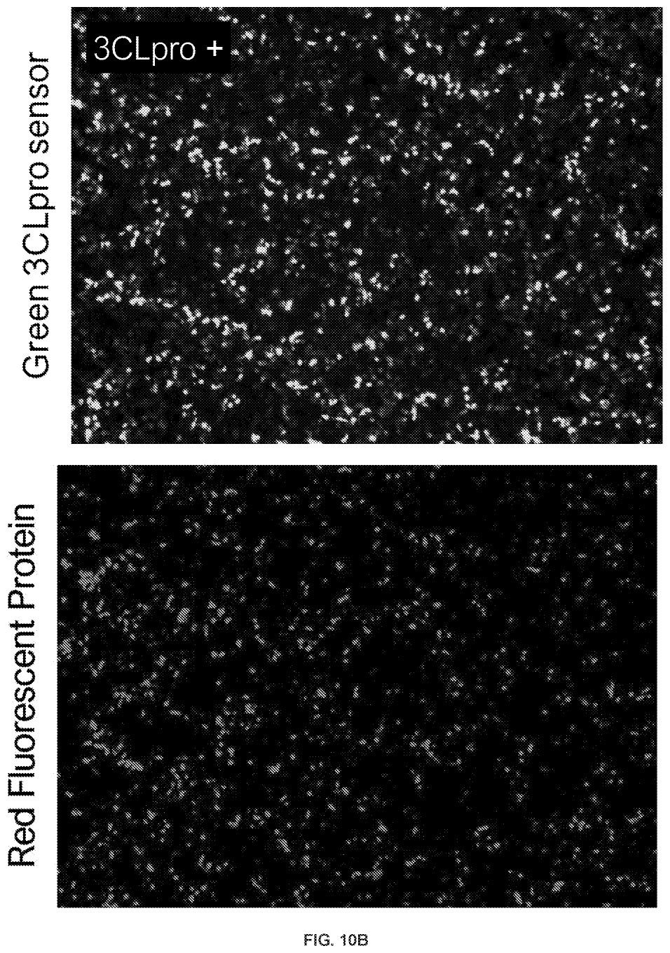

HEK 293 cells were transduced with BacMam vectors expressing the two-reporter (green and red) 3CLpro sensor of Example 5. All of the cells showed red fluorescent signal, indicating they were healthy and providing a fluorescent signal showing how many were present. Only the cells that were co-transduced with a second BacMam vector expressing the 3CLpro enzyme showed a green signal to any significant extent. As expected, the green signal was dependent on the expression of the 3CLpro enzyme. FIG. 10A shows cells transfected with only the two-reporter vector. FIG. 10B shows cells transfected with both the two-reporter vector and the second BacMam vector encoding the 3CLpro enzyme.

### Example 7: Detection of 3CL Protease Inhibition

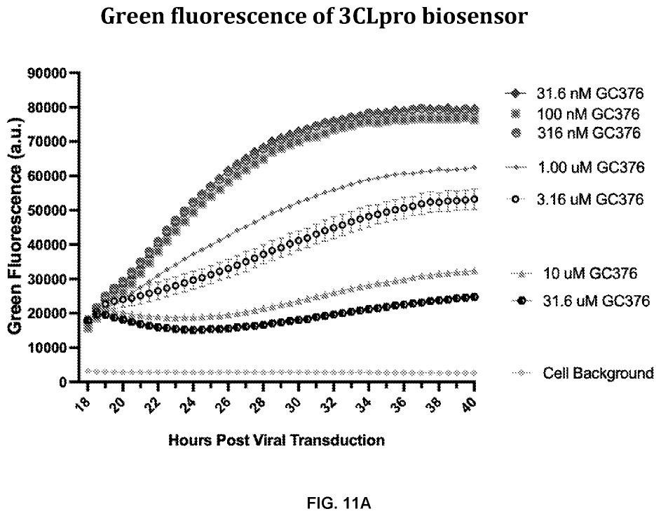

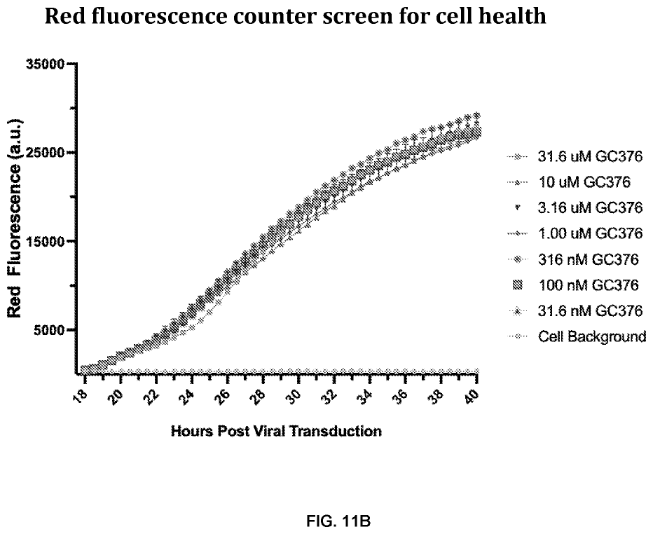

The combined expression of the 3CLpro and the 3CLpro biosensor of Example 5 can be used in live cell assays to identify 3CLpro inhibitors. FIGS. 11A and 11B show BacMam vector delivery used to co-express the SARS-CoV-2 protease, a green fluorescent 3CLpro biosensor, and a consecutively expressed red fluorescent protein. HEK 293 cells were also treated with varying amounts of 3CL protease inhibitor GC376. Cells were plated on day one in a 96 well tissue culture plate. The following day the BacMam viruses were added. Eighteen hours after the transduction, the inhibitor GC376 was added to different wells, at different concentrations, to measure the dose/response relationship between the inhibitor and the green fluorescence of the 3CLpro biosensor. The fluorescence of each well was measured with a Synergy fluorescence plate reader available from Biotek, Vermont, USA. At very low concentrations of inhibitor, the green fluorescence rose quickly over time, indicating significant 3CLpro activity in the living cells. At higher concentrations of the inhibitor, the green fluorescence was much weaker. This diminution of the fluorescence signal was not due to cell death, as the signal from the constitutively expressed red fluorescent protein continued to increase for all transduced cells. FIGS. 11A and 11B also show un-transduced cells.

### Example 8: Detection of 3CL Protease Inhibition via a Normalized Fluorescent Signal

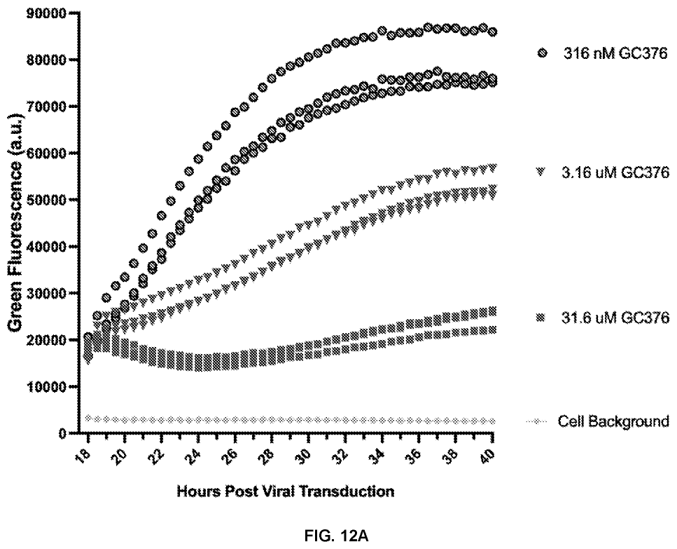

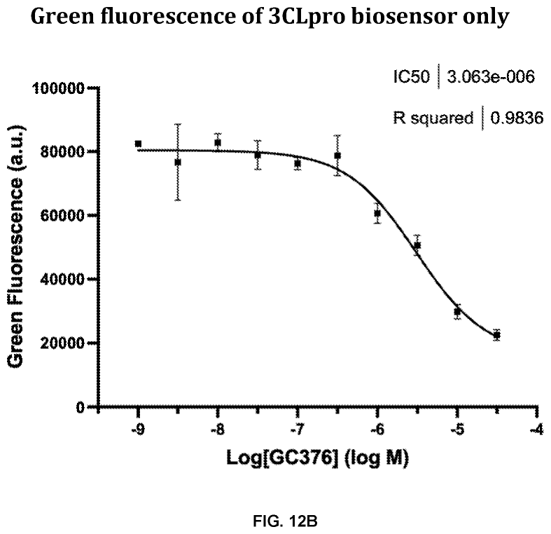

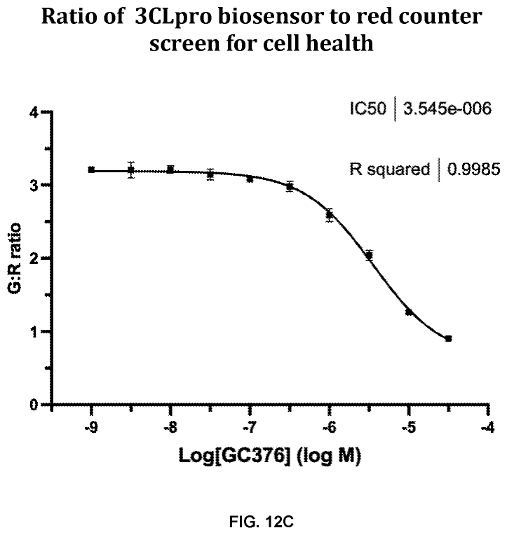

Cells were transduced with the 3CLpro biosensor of Example 5 and the 3CL protease. Inspection of the green fluorescence signal generated over time in individual wells demonstrates that much of the variability in the measurement of the protease activity at any point in time, at any particular concentration of the inhibitor, is due to systematic differences between wells. This is illustrated in FIG. 12A, where three different wells are plotted individually for each of three different concentrations of the inhibitor GC376 (Vuong et al., 2020. Nat. Commun. 11, 4282). This well-to-well variability produces a significant standard deviation at any particular concentration of the inhibitor (FIG. 12B), particularly at very low concentrations of the inhibitor when the 3CLpro may affect cell health. Ratio measurements of the green fluorescence produced by the 3CLpro sensor, and the constitutively expressed red fluorescent protein, significantly lower the standard deviation at any particular inhibitor concentration (FIG. 12C) by accounting for well-to-well differences in the number of healthy cells.

### Example 9: Detection and Characterization of a Novel 3CL Protease Inhibitor

FIGS. 13A and 13B show results from a 3CLpro assay using the biosensor of Example 5 and performed in HEK 293 cells. The assay tested different concentrations of a new putative inhibitor (Compound 43) or the known inhibitor GC376. Compound 43 has nM efficacy against recombinant 3CLpro in a biochemical assay. However, compound 43 potency is far less in living HEK 293 cells, where the 3CLpro assay shows very low efficacy and a micromolar IC₅₀ (FIG. 13A, triangles). One explanation for this could be that the P-glycoprotein multidrug transporter lowers the intracellular concentration of the compound (Sharom, 2011. Essays Biochem. 50, 161-178). Indeed, the addition of the transporter inhibitor CP100356 (CP, circles) changes the efficacy of the compound (FIG. 13A). In contrast, inhibiting the P-glycoprotein multidrug transporter has no effect on the efficacy of GC376, indicating that it is not pumped out of the cell (FIG. 13B). This illustrates how a live cell assay for 3CLpro can be used to examine the effect of a potential inhibitor in the context of physiologically relevant, living cells.

### Example 10: Detection of 3CL Protease Inhibition

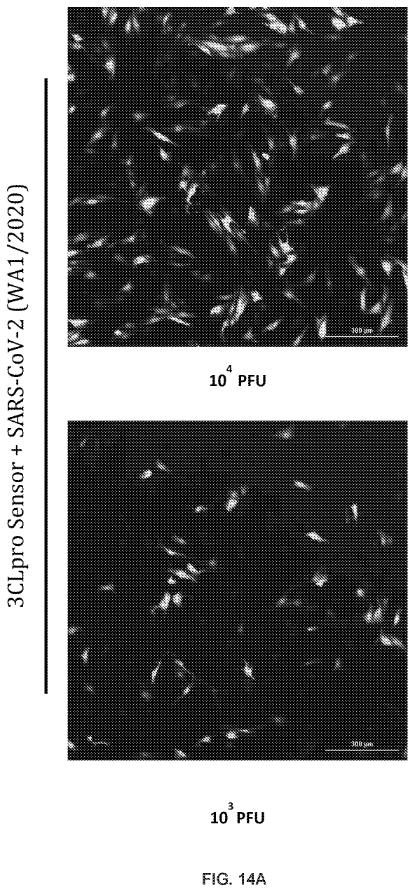

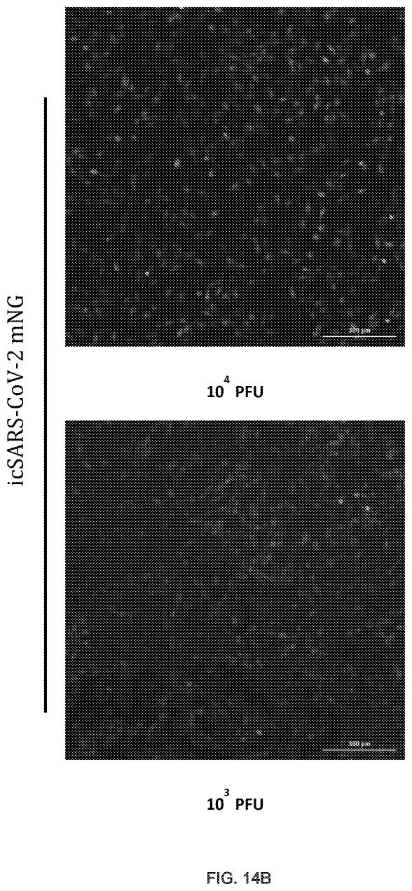

FIG. 14A shows fluorescence micrographs of Vero E6 cells that were transduced on the first day with the BacMam vector expressing the 3CLpro biosensor of Example 5. The following day, live SARS-CoV-2 virus was added to the wells at two different MOI (Plaque Forming Units, PFU). The 3CLpro biosensor reported virus replication in these cells by producing bright green fluorescence, and the number of fluorescent cells was consistent with the amount of SARS-CoV-2 virus added to the well (compare two panels). FIG. 14B shows Vero E6 cells in adjacent wells transduced with the recombinant icSARS-CoV-2 mNeonGreen virus (Xie et al., 2020. Cell Host Microbe 27, 841-848.e3). Here, the signal is much weaker and difficult to detect. This difference illustrates one example of the benefits of the biosensor of the present disclosure. The biosensor is able to produce higher amounts of fluorescent protein, uncoupled from coronaviral genome expression and replication, in contrast to the construct tested in FIG. 14B.

1. A vector comprising a nucleic acid comprising:
a nucleotide sequence comprising a 5′ untranslated region,
a nucleotide sequence encoding a degron,
a nucleotide sequence encoding a cleavage site, and
a nucleotide sequence encoding a reporter protein.

2-3. (canceled)

4. The vector of claim 1, wherein the 5′ untranslated region comprises a 5′ untranslated region of the SARS-CoV-2 virus genome.

5. The vector of claim 1, wherein the degron comprises a ubiquitin domain, optionally wherein the ubiquitin domain comprises the amino acid sequence of SEQ ID NO: 4.

6. (canceled)

7. The vector of claim 1, wherein the cleavage site is specifically cleaved by 3C-like protease, optionally wherein the cleavage site comprises the amino acid sequence of SEQ ID NO: 6.

8. (canceled)

9. The vector of claim 1, wherein the vector comprises a nucleotide sequence that is at least 75% identical to a nucleotide sequence selected from the group consisting of SEQ ID NOs: 7-9, 23 and 24.

10-12. (canceled)

13. The vector of claim 1, wherein the cleavage site is specifically cleaved by papain-like protease or by a caspase.

14. (canceled)

15. The vector of claim 1, wherein the reporter protein comprises a fluorescent protein, optionally wherein the fluorescent protein comprises mNeonGreen or Red Fluorescent Protein.

16-17. (canceled)

18. The vector of claim 1,
wherein the nucleotide sequence comprising the 5′ untranslated region comprises a nucleotide sequence that is at least 75%, 80%, 85%, 90%, 95%, 96%, 97%, 98%, 99% or 100% identical to nucleotides 1,613-1,877 of SEQ ID NO: 11,
wherein the degron comprises an amino acid sequence that has 0, 1, 2, 3, 4 or 5 amino acid changes compared to the amino acid sequence of SEQ ID NO: 4, and
wherein the cleavage site comprises an amino acid sequence that has 0, 1, 2, 3, 4 or 5 amino acid changes compared to the amino acid sequence of SEQ ID NO: 6.

19. The vector of claim 1,
wherein the reporter protein comprises an amino acid sequence that is at least 75% identical to SEQ ID NO: 13 or 14.

20. A vector comprising a nucleic acid comprising
a nucleotide sequence encoding a 5′ untranslated region,
a nucleotide sequence encoding a degron,
a nucleotide sequence encoding a cleavage site,
a nucleotide sequence encoding a first reporter protein, and
a nucleotide sequence encoding a second reporter protein.

21-35. (canceled)

36. The vector of claim 20, further comprising a self-cleaving peptide encoded by nucleotides that are positioned between the nucleotides encoding the first reporter protein and the nucleotides encoding second reporter protein or a self-cleaving peptide encoded by nucleotides that are positioned between the nucleotides encoding the degron and the nucleotides encoding second reporter protein.

37. (canceled)

38. The vector of claim 36 or 37, wherein the self-cleaving peptide, if completely translated, would comprise the amino acid sequence of SEQ ID NO: 15.

39-40. (canceled)

41. The vector of claim 1, wherein the vector is packaged in a baculovirus, optionally wherein the baculovirus is BacMam.

42-45. (canceled)

46. The vector of claim 1, wherein the vector comprises a nucleic acid comprising the sequence of positions 1614 to 2208 of any one of SEQ ID NO: 7, SEQ ID NO: 8, and SEQ ID NO: 9.

47. A biosensor encoded by the vector of claim 1.

48. A cell comprising the vector of claim 1.

49. (canceled)

50. A method for detecting protease activity in a cell comprising measuring a signal from the biosensor of claim 47.

51. (canceled)

52. A method of detecting SARS-CoV-2 infection in a sample from a subject, wherein the sample comprises cells from the subject, comprising introducing an effective amount of the vector of claim 1 to the cells in the sample and measuring a signal from the reporter protein.

53. A method of detecting a protease inhibitor specific for a protease present in a cell comprising introducing an effective amount of the vector of claim 1 to the cell and measuring a signal from the reporter protein.

54-56. (canceled)

57. A method of measuring replication of a virus that comprises a protease in a cell comprising introducing an effective amount of the vector of claim 1 to the cell and measuring a signal from the reporter protein.

58. (canceled)


SEQUENCE LISTING (25 sequences)

SEQ ID NO: 1 - DNA, 918 bp, synthetic construct
  misc_feature 1..918: note = 3CL Protease Sequence
  source 1..918: mol_type = other DNA
tctggttttaggaaaatggcgttccccagcggtaaagttgaaggatgtatggtccaagtaacctgtggtaccactacccttaatgggctttggttggacgacgtagtctactgcccccgacacgtaatctgcactagtgaggatatgcttaatcccaattacgaagaccttttgattcggaaatccaatcacaacttcctggtccaagcgggcaacgtccaactcagggttattggacatagtatgcagaattgcgtactgaagctcaaagtcgatactgcaaaccccaagacgcccaagtataaattcgtccgaatccaaccaggccaaacattttccgtattggcttgctataatggaagccccagcggtgtctaccaatgtgcaatgagaccaaactttacgataaagggttcatttctgaacggctcttgcggttccgttggttttaacatcgactatgactgtgtatccttttgctacatgcaccatatggaactccctaccggtgtccacgccggtacagatctggaaggaaatttctacggtccgttcgttgaccggcaaaccgcgcaagcggctggaaccgacacaacgattacagtgaatgtgctcgcgtggctgtacgcagcagtcataaacggagacaggtggtttctgaaccgatttacgacgactctcaatgacttcaaccttgttgcgatgaagtacaattacgagccactcacccaggaccatgttgatatcctgggtcccctcagtgcccagacagggatcgcagttctcgatatgtgcgcgtcactgaaggagcttctccaaaatggaatgaatgggcggaccatacttggttccgcactcctcgaagatgaatttactccatttgacgtggtcagacaatgcagtggggtcactttccag

SEQ ID NO: 2 - DNA, 2909 bp, synthetic construct
  misc_feature 1..2909: note = 3CL protease construct
  source 1..2909: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatacgactcactatagggagacccaagctggctagcgtttgccgccaccatgtctggttttaggaaaatggcgttccccagcggtaaagttgaaggatgtatggtccaagtaacctgtggtaccactacccttaatgggctttggttggacgacgtagtctactgcccccgacacgtaatctgcactagtgaggatatgcttaatcccaattacgaagaccttttgattcggaaatccaatcacaacttcctggtccaagcgggcaacgtccaactcagggttattggacatagtatgcagaattgcgtactgaagctcaaagtcgatactgcaaaccccaagacgcccaagtataaattcgtccgaatccaaccaggccaaacattttccgtattggcttgctataatggaagccccagcggtgtctaccaatgtgcaatgagaccaaactttacgataaagggttcatttctgaacggctcttgcggttccgttggttttaacatcgactatgactgtgtatccttttgctacatgcaccatatggaactccctaccggtgtccacgccggtacagatctggaaggaaatttctacggtccgttcgttgaccggcaaaccgcgcaagcggctggaaccgacacaacgattacagtgaatgtgctcgcgtggctgtacgcagcagtcataaacggagacaggtggtttctgaaccgatttacgacgactctcaatgacttcaaccttgttgcgatgaagtacaattacgagccactcacccaggaccatgttgatatcctgggtcccctcagtgcccagacagggatcgcagttctcgatatgtgcgcgtcactgaaggagcttctccaaaatggaatgaatgggcggaccatacttggttccgcactcctcgaagatgaatttactccatttgacgtggtcagacaatgcagtggggtcactttccagtaacgcgccctcgactgtgccttctagttgccagccatctgtatcgcggccgctctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgg

SEQ ID NO: 3 - DNA, 2909 bp, synthetic construct; identical to SEQ ID NO: 2
  misc_feature 1..2909: note = ubiquitin sequence
  source 1..2909: mol_type = other DNA

SEQ ID NO: 4 - protein, 76 aa, synthetic construct [sequence omitted]

SEQ ID NO: 5 - DNA, 30 bp, synthetic construct
  misc_feature 1..30: note = 3CL protease target sequence
  source 1..30: mol_type = other DNA
acagtacgtctacaagctggtaatgcaaca

SEQ ID NO: 6 - protein, 10 aa, synthetic construct
  REGION 1..10: note = 3CL protease target sequence
TVRLQAGNAT

SEQ ID NO: 7 - DNA, 3303 bp, synthetic construct
  misc_feature 1..3303: note = exemplary construct
  source 1..3303: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatacgactcactatagggagacccaagctggctagcgtttattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatgcagatcttcgtgaagaccctgaccggcaagaccatcaccctggaggtggagcccagcgacaccatcgagaacgtgaaggccaagatccaggacaaggagggcatcccccccgaccagcagaggctgatcttcgccggcaagcagctggaggacggcaggaccctgagcgactacaacatccagaaggagagcaccctgcacctggtgctgaggctgaggggcggcgagaatagaggtatggtacttggtagtttagctgccacagtacgtctacaagctggtaatgcaacagaagtgcctgccaattcaactgtattatctttctgtatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagcctaagaagaagaggaaggtctaacgcgccctcgactgtgccttctagttgccagccatctgtatcgcggccgctctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgg

SEQ ID NO: 8 - DNA, 3282 bp, synthetic construct
  misc_feature 1..3282: note = exemplary construct
  source 1..3282: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatacgactcactatagggagacccaagctggctagcgtttattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatgcagatcttcgtgaagaccctgaccggcaagaccatcaccctggaggtggagcccagcgacaccatcgagaacgtgaaggccaagatccaggacaaggagggcatcccccccgaccagcagaggctgatcttcgccggcaagcagctggaggacggcaggaccctgagcgactacaacatccagaaggagagcaccctgcacctggtgctgaggctgaggggcggctataatagaggtatggtacttggtagtttagctgccacagtacgtctacaagctggtaatgcaacagaagtgcctgccaattcaactgtattatctttctgtatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagaatcgcgccctcgactgtgccttctagttgccagccatctgtatcgcggccgctctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgg

SEQ ID NO: 9 - DNA, 3282 bp, synthetic construct
  misc_feature 1..3282: note = exemplary construct
  source 1..3282: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatacgactcactatagggagacccaagctggctagcgtttattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatgcagatcttcgtgaagaccctgaccggcaagaccatcaccctggaggtggagcccagcgacaccatcgagaacgtgaaggccaagatccaggacaaggagggcatcccccccgaccagcagaggctgatcttcgccggcaagcagctggaggacggcaggaccctgagcgactacaacatccagaaggagagcaccctgcacctggtgctgaggctgaggggcggcaggaatagaggtatggtacttggtagtttagctgccacagtacgtctacaagctggtaatgcaacagaagtgcctgccaattcaactgtattatctttctgtatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagaatcgcgccctcgactgtgccttctagttgccagccatctgtatcgcggccgctctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgg

SEQ ID NO: 10 - protein, 306 aa, synthetic construct [sequence omitted]

SEQ ID NO: 11 - DNA, 4124 bp, synthetic construct
  misc_feature 1..4124: note = exemplary construct
  source 1..4124: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatacgactcactatagggagacccaagctggctagcgtttattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatgcagatcttcgtgaagaccctgaccggcaagaccatcaccctggaggtggagcccagcgacaccatcgagaacgtgaaggccaagatccaggacaaggagggcatcccccccgaccagcagaggctgatcttcgccggcaagcagctggaggacggcaggaccctgagcgactacaacatccagaaggagagcaccctgcacctggtgctgaggctgaggggcggcaggaatagaggtatggtacttggtagtttagctgccacagtacgtctacaagctggtaatgcaacagaagtgcctgccaattcaactgtattatctttctgtatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggacgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaaggagggcagaggaagtcttctaacatgcggtgacgtggaggagaatcccgggcctatggtgtcgaagggagaagaactgattaaggaaaacatgcggatgaaagtggtgatggaagggtcggtgaatggacaccaattcaagtgcaccggagagggagagggcaacccatacatgggtactcagaccatgcgcatcaaggtcgttgaaggaggacctctgccttttgcgttcgacatccttgctacctcgttcatgtacgggtcccgcacctttatcaagtacccgaagggaatcccggatttcttcaagcagagcttcccggaaggattcacctgggagagggtgactcggtacgaagatggaggagtgctgactgcaacccaagacacttcgctcgaggacggctgtctggtgtaccatgtccaagtgcggggtgtgaacttcccctcaaatgggccagtgatgcagaaaaagaccctcggatgggaagcgaacaccgagatgatgtacccggcggacggtggcttgcgaggatacactcacatggccttgaagctggacggcggaggtcatctctcatgctcctttgtcactacctaccgcagcaagaaaactgtcggaaacatcaagatgccgggcgtgtactacgtcgatcaccggctcgagagaatcaaagaggccgacaaggaaacgtatgtcgagcagcatgaagtcgcagtggccaggtactgcgaccttccctccaaactgggccacaagctgaattctggcctgagaagccgcgcacaggcttcgaactcagccgtggatgggacggccggcccagggtccactggaagcagataacgcgccctcgactgtgccttctagttgccagccatctgtatcgcggccgctctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgg

SEQ ID NO: 12 - protein, 319 aa, synthetic construct
  REGION 1..319: note = SARS-CoV-2 PL protease
EVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNSYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTY

SEQ ID NO: 13 - protein, 236 aa, synthetic construct
  REGION 1..236: note = exemplary reporter protein
MVSKGEEDNMASLPATHELHIFGSINGVDFDMVGQGTGNPNDGYEELNLKSTKGDLQFSPWILVPHIGYGFHQYLPYPDGMSPFQDAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 14 - protein, 263 aa, synthetic construct
  REGION 1..263: note = exemplary reporter protein
MVSKGEELIKENMRMKVVMEGSVNGHQFKCTGEGEGNPYMGTQTMRIKVVEGGPLPFAFDILATSFMYGSRTFIKYPKGIPDFFKQSFPEGFTWERVTRYEDGGVLTATQDTSLEDGCLVYHVQVRGVNFPSNGPVMQKKTLGWEANTEMMYPADGGLRGYTHMALKLDGGGHLSCSFVTTYRSKKTVGNIKMPGVYYVDHRLERIKEADKETYVEQHEVAVARYCDLPSKLGHKLNSGLRSRAQASNSAVDGTAGPGSTGSR

SEQ ID NO: 15 - protein, 18 aa, synthetic construct
  REGION 1..18: note = exemplary self-cleaving peptide signal
EGRGSLLTCGDVEENPGP

SEQ ID NO: 16 - DNA, 160 bp, synthetic construct
  misc_feature 1..160: note = exemplary partial biosensor sequence
  source 1..160: mol_type = other DNA
acctggtgctgaggctgaggggcggcaggaatagaggtatggtacttggtagtttagctgccacagtacgtctacaagctggtaatgcaacagaagtgcctgccaattcaactgtattatctttctgtatggtgagcaagggcgaggaggataacatggc

SEQ ID NO: 17 - protein, 54 aa, synthetic construct
  REGION 1..54: note = exemplary partial biosensor sequence
HLVLRLRGGRNRGMVLGSLAATVRLQAGNATEVPANSTVLSFCMVSKGEEDNMA

SEQ ID NO: 18 - protein, 4 aa, synthetic construct
  REGION 1..4: note = exemplary cleavage site sequence
DVED

SEQ ID NO: 19 - protein, 4 aa, synthetic construct
  REGION 1..4: note = exemplary cleavage site sequence
WEHD

SEQ ID NO: 20 - protein, 5 aa, synthetic construct
  REGION 1..5: note = exemplary cleavage site sequence
VDVAD

SEQ ID NO: 21 - protein, 4 aa, synthetic construct
  REGION 1..4: note = exemplary cleavage site sequence
LEVD

SEQ ID NO: 22 - protein, 4 aa, synthetic construct
  REGION 1..4: note = exemplary cleavage site sequence
LETD

SEQ ID NO: 23 - DNA, 595 bp, synthetic construct
  misc_difference 1..595: note = exemplary biosensor partial sequence
  misc_difference 494..529: note = n is a, c, g, or t
  misc_difference 560..595: note = n is a, c, g, or t
  source 1..595: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatgcagatcttcgtgaagaccctgaccggcaagaccatcaccctggaggtggagcccagcgacaccatcgagaacgtgaaggccaagatccaggacaaggagggcatcccccccgaccagcagaggctgatcttcgccggcaagcagctggaggacggcaggaccctgagcgactacaacatccagaaggagagcaccctgcacctggtgctgaggctgaggggcggcnnnnnnnnnnnnnnnnnnnnnnnnnnnnnnnnnnnnacagtacgtctacaagctggtaatgcaacannnnnnnnnnnnnnnnnnnnnnnnnnnnnnnnnnnn

SEQ ID NO: 24 - DNA, 330 bp, synthetic construct
  misc_difference 1..330: note = exemplary biosensor partial sequence
  misc_difference 229..264: note = n is a, c, g, or t
  misc_difference 295..330: note = n is a, c, g, or t
  source 1..330: mol_type = other DNA
atgcagatcttcgtgaagaccctgaccggcaagaccatcaccctggaggtggagcccagcgacaccatcgagaacgtgaaggccaagatccaggacaaggagggcatcccccccgaccagcagaggctgatcttcgccggcaagcagctggaggacggcaggaccctgagcgactacaacatccagaaggagagcaccctgcacctggtgctgaggctgaggggcggcnnnnnnnnnnnnnnnnnnnnnnnnnnnnnnnnnnnnacagtacgtctacaagctggtaatgcaacannnnnnnnnnnnnnnnnnnnnnnnnnnnnnnnnnnn

SEQ ID NO: 25 - protein, 110 aa, synthetic construct
  REGION 1..110: note = exemplary biosensor partial sequence
  VARIANT 77..88: note = X can be any naturally occurring amino acid
  VARIANT 99..110: note = X can be any naturally occurring amino acid
MQIFVKTLTGKTITLEVEPSDTIENVKAKIQDKEGIPPDQQRLIFAGKQLEDGRTLSDYNIQKESTLHLVLRLRGGXXXXXXXXXXXXTVRLQAGNATXXXXXXXXXXXX